(19) United States
(12) Patent Application Publication
(10) Pub. No.: US 2016/0120833 A1
(43) Pub. Date: May 5, 2016

(54) COMPOSITION AND METHOD FOR TREATING AND RELIEVING MYOPIA

(71) Applicants: SUNDRAGON BIOMEDICAL ELECTRONICS CO., LTD., Taichung (TW); CHINA MEDICAL UNIVERSITY, Taichung (TW)

(72) Inventors: Lei WAN, TAICHUNG (TW); Hui-Ju LIN, TAICHUNG (TW)

(21) Appl. No.: 14/926,519

(22) Filed: Oct. 29, 2015

Related U.S. Application Data
(60) Provisional application No. 62/073,037, filed on Oct. 31, 2014.

(51) Int. Cl. A61K 31/216; A61K 9/00; A61K 9/08; A61K 31/05; A61K 31/196

(52) U.S. Cl. CPC A61K 31/216; A61K 31/05; A61K 31/196; A61K 9/08; A61K 9/0048

(57) ABSTRACT

The present invention provides a pharmaceutical composition for treating and/or relieving myopia, the pharmaceutical composition comprises a therapeutically effective amount of an anti-inflammatory agent and a pharmaceutically acceptable carrier; the pharmaceutical composition of the present invention is safe, and can treat and/or relieve myopia by the anti-inflammatory agent. The pharmaceutically acceptable carrier can effectively encapsulate the anti-inflammatory agent at a specific ratio, and the stability and solubility of the pharmaceutical composition can be enhanced.

### BACKGROUND OF THE INVENTION

1. Field of the Invention

The present invention relates to a pharmaceutical composition, especially a composition comprising an anti-inflammatory drug and a pharmaceutically acceptable carrier at a specific weight ratio. The present invention relates to a method for preparing the aforementioned pharmaceutical composition, especially by encapsulating anti-inflammatory drugs by the pharmaceutically acceptable carrier. The present invention further relates to a method for treating and/or relieving myopia.

2. Description of the Prior Arts

The prevalence of myopia has rapidly increased in recent decades and has led to a considerable global public health concern. Globally, there are approximately 153 million people over the age of 5 years who suffer from visual defects, 8 million of whom suffer from blindness because of uncorrected myopia and other refractive errors (REs). In the United States alone, the economic costs of myopia have increased to US$250 million per year. Myopia is a prominent and often undertreated eye disease. Although most cases of myopia can be corrected with glasses, contact lenses, or refractive surgery, uncorrected REs still account for approximately 33% of visual impairments. High-degree myopia is a particularly dangerous visual affliction because of the higher risks of macular and retinal complications. Myopia results primarily from abnormal elongation of the vitreous chamber of the eye. This condition is recapitulated in the monocular form deprivation (MFD) animal model, which has been used to study myopia pathogenesis. Eye elongation is associated with remodeling of the sclera, loss of scleral tissue through reduced connective tissue synthesis, and increased collagen I (COL1) degradation, resulting in changes in the composition and ductility of the sclera. Recent studies in monkeys have demonstrated that the retina, specifically, photoreceptors and retinal pigment epithelium, plays a crucial role in modulating eye growth and axial length through producing activating signals that promote scleral tissue remodeling.

Animal studies of myopia have shown that atropine—a nonselective muscarinic acetylcholine receptor (mAChR) antagonist—effectively prevents axial elongation, which leads to myopia. Atropine inhibits myopia progression in tree shrews, monkeys, chickens, guinea pigs, rats, mice, and Syrian hamsters, and its effectiveness has also been demonstrated in human clinical trials. However, the mechanism of this effect remains unclear. Five distinct genes (CHRM1-5) encode the 7-transmembrane mAChR proteins M1 to 5, each of which has distinct pharmacological properties. Our previous studies showed that CHRM1 and 3 are predominantly responsible for the changes in axial elongation associated with myopia, and CHRM3 plays a crucial role in myopia pathogenesis as well as in the atropine-mediated block of myopia progression.

Various molecules have been implicated in myopia progression. In myopic eyes, transforming growth factor-β (TGF-β) and matrix metalloproteinase 2 (MMP2) expression is elevated, whereas COL1 expression is downregulated. 25 TGF-θ regulates cellular functions such as cell growth, differentiation, inflammation, and wound healing, whereas MMP family members play major roles in the breakdown of the extracellular matrix, tissue reconstruction, and vascularization during the inflammatory response. The dysregulation of MMPs has been proposed as a mechanism of pathogenesis in myopic eyes; MMP2 expression is upregulated in the sclera of chickens and tree shrews in which myopia has been induced through form deprivation. TGF-β regulates the level of MMP2 through activation of nuclear factor-κB (NF-κB), a transcription factor that modulates the expression of various inflammatory cytokines in fibroblasts.

Several reports have proposed the role of inflammation in myopia progression. Uveitis can induce acute or constitutive myopia and myopic shift, and acute myopia has been observed in patients with acute scleritis. A 26-year follow-up of patients with juvenile chronic arthritis (JCA) revealed myopic REs in a greater proportion of these patients than in age-matched control patients, suggesting a correlation between JCA and myopia. The same study suggested that the higher incidence of myopia was due to the weakening of scleral connective tissue as a result of chronic inflammation. In addition, acute-onset myopia may be a presenting feature of systemic lupus erythematosus (SLE). The experimental and clinical evidence from the present study indicates a direct link between inflammation and myopia progression.

### SUMMARY OF THE INVENTION

To overcome the shortcomings of lack of commercially available drugs for myopia treatment, the objective of the present invention is to provide a pharmaceutical composition for use in treating and/or relieving myopia, comprising a therapeutically effective amount of an anti-inflammatory agent and a pharmaceutically acceptable carrier, wherein the anti-inflammatory agent comprises resveratrol, diacerein or diclofenac.

Preferably, the pharmaceutically acceptable carrier comprises potyoxyethylene castor oil ether (Cremophor EL), alkyldimethylbenzylammonium (BKC), lecithin, cholesterol, Dulbecco's phosphate buffered saline (DPBS), cyclodextrin, tween 80, castor oil, artificial tears, and the any combination thereof.

According to the present invention, the term “therapeutically effective amount” as used herein, refers to a dosage to treat or relieve myopia. The therapeutically effective amount for treating or relieving myopia is determined by administering the pharmaceutical composition in an effective amount, and measuring the MMP2, TGF-β, NF-κB, c-Fos, TNF-α, Il-6, or IL10 expression in a specific period.

According to the present invention, the term “artificial tears” as used herein, refers to human tears; the ingredients include, but are not limited to water, salt, sodium hyaluronate, carboxymethyl cellulose, hydroxypropyl methyl cellulose or hydroxypropyl cellulose prime; the use of the artificial tears of the present invention are known to a skilled person in the art, such as for distributing water upon the surface of the eye to moisture eyes, and alleviating eyestrain.

In another preferred embodiment, the anti-inflammatory agent is resveratrol, the concentration of resveratrol is between 0.1 M and 0.2 M, and the pharmaceutically acceptable excipient comprises cyclodextrin and artificial tears.

Preferably, the pharmaceutical composition comprises 0.1141 g resveratrol, 1.46 g cyclodextrin and 1 ml artificial tears.

In still another preferred embodiment, the anti-inflammatory agent is diacerein, the concentration of diacerein is between 0.01 M and 0.02 M, and the pharmaceutically acceptable excipient comprises tween 80, castor oil and artificial tears.

Preferably, the volume ratio of tween 80 to castor oil is 0.01 to 0.02:0.003 with a total volume of artificial tears.

Preferably, the pharmaceutical composition comprises 0.004 g diacerein, 1 ml artificial tears, 10 μl tween 80 and 3 μl castor oil.

In further another preferred embodiment, the anti-inflammatory agent is diclofenac, the concentration of diclofenac is between 0.01 M and 0.02 M, and the pharmaceutically acceptable excipient comprises artificial tears.

Preferably, the pharmaceutical composition comprises 0.004 g diclofenac and 1 ml artificial tears.

Preferably, the pharmaceutical composition may include a pharmaceutically acceptable carrier, wherein examples of the carrier include water, saline, phosphate buffered saline, dextrose, glycerol, ethanol and one or more combinations thereof. The pharmaceutically acceptable carrier may further include auxiliary substances such as wetting or emulsifying agents, preservatives or buffers.

The present invention further provides a method for preparing said pharmaceutical composition comprising:

preparing the anti-inflammatory agent;

preparing the pharmaceutically acceptable carrier; and

adding the therapeutically effective amount of the anti-inflammatory agent gradually into the pharmaceutically acceptable carrier under an oscillating condition to obtain the pharmaceutical composition.

Preferably, the anti-inflammatory agent is resveratrol, the pharmaceutically acceptable excipient comprises cyclodextrin and artificial tears; the resveratrol is enveloped by cyclodextrin, a molar ratio of resveratrol to cyclodextrin is 1:2 to form a cladding, and the cladding is added to artificial tears at a weight ratio of 0.01 to 0.03:1 progressively and gradely to obtain the pharmaceutical composition.

Preferably, the anti-inflammatory agent is diacerein, the pharmaceutically acceptable excipient comprises tween 80, castor oil and artificial tears; the volume ratio of tween 80 to castor oil is 0.01 to 0.02:0.003 with a total volume of artificial tears to form a mixture, and then diacerein is added to the mixture progressively and gradely to obtain the pharmaceutical composition.

Preferably, the anti-inflammatory agent is diclofenac, the pharmaceutically acceptable excipient comprises artificial tears; diacerein is added to the mixture progressively and gradely with the weight ratio of diclofenac to artificial tears being 0.03 to 0.06:0.001 to obtain the pharmaceutical composition.

The present invention further provides a method for treating and/or relieving myopia comprising a step of administering to a subject in need thereof a therapeutically effective amount of the above-said pharmaceutical composition.

Preferably, the subject is animal or human.

In accordance with the present invention, the pharmaceutical composition for treating and/or relieving myopia is prepared in multiple forms, including, but not limited to, liquid, semi-solid and solid dosage, wherein the liquid solution includes injectable and infusible solution, dispersions or suspensions, wherein the solid dosage includes tablets, pills, powders, liposomes and suppositories. Preferred form depends on the mode of administration and therapeutic application of expectations. Preferably, the pharmaceutical composition of the present invention is administered orally, by topical injection or in the form of external use. Preferably, the more preferred embodiment of the external use includes, but is not limited to, perfusion ointment, patch, subcutaneous implants, drops or gel. More preferably, the pharmaceutical composition is manufactured to be suitable for applying to eye in topically external formulation, whose dosage form includes, but is not limited to, ointments, drops or gels.

More preferably, the pharmaceutical composition of the present invention as an external formulation may further comprise additives, wherein the additives include, but are not limited to, preserving agents, antioxidants, surfactants, absorption enhancers, stabilizing agents, active agents, humectants, pH adjusting agents, solubilizing agents, penetration enhancers and anti-irritants agents; species selection and the amount of the above additives are familiar to those skilled in the art.

Preferably, the therapeutically effective amount of the pharmaceutical composition for treating and/or relieving myopia in external dosage form is between 0.5% and 1%.

Preferably, the therapeutically effective amount of the pharmaceutical composition for treating and/or relieving myopia in oral administration form is between 10 mg/day and 50 mg/day.

The pharmaceutical composition of the present invention can be used for inhibiting inflammation and for treating and relieving the progression of myopia. The pharmaceutical composition of the present invention is safe and non-toxic to normal cells. Furthermore, the pharmaceutical composition of the present invention still can treat and relieve the inflammatory eyes in the effective therapeutic amount. In addition, the pharmaceutically acceptable carrier can effectively encapsulate the anti-inflammatory agent at a specific ratio, and the stability and the solubility of the pharmaceutical composition could be enhanced.

### BRIEF DESCRIPTION OF THE DRAWINGS

The patent or application file contains at least one drawing executed in color. Copies of the patent or patent application publication with color drawing(s) will be provided by the Office upon request and payment of the necessary fee.

FIG. 1A shows that the RE was determined as the difference between the diopter measurements recorded after (F) and before (B), wherein “BR” means right eye before MFD; wherein “BL” means left eye before MFD; wherein “FR” means right eye after MFD (occluded eye); wherein “FL” means left eye after MFD. A Student's t test was used for paired comparisons between FR-BR and FL-BL; P<0.05 was considered statistically significant. FIG. 1B is an immunohistochemical analysis of CHRM1 and CHRM3. FIG. 1C is an immunohistochemical analysis of MMP2 and COL1. FIG. 1D is an immunohistochemical analysis of TGF-β in sclera and retina.

FIG. 2A shows that the RE was determined as the difference between the diopter measurements recorded after (F) and before (B) MFD in hamsters treated for 21 days with 3% CSA. FIG. 2B is an immunohistochemical analysis of TGF-β and MMP2 expression in control eyes (control [L]), MFD eyes (control [R]), 3% CSA-treated control eyes (CSA [L]), and 3% CSA-treated MFD eyes (CSA [R]).

FIG. 3A shows that the refractive error (RE) was determined as the difference between the diopter measurements recorded after (F) and before (B) MFD in hamsters treated for 21 days with LPS or PGN. FIG. 3B is an immunohistochemical analysis of TGF-β and MMP2 expression in control eyes ([L]) and form-deprived MFD eyes ([R]) with or without LPS or PGN. FIG. 3C shows that the RE was determined as the difference between the diopter measurements in hamsters treated for 21 days with LPS or PGN. FIG. 3D is an immunohistochemical analysis of MMP2, collagen I, and TGF-β expression in eyes with or without LPS or PGN.

FIGS. 4A and 4D are immunohistochemical analysis of c-Fos, NF-Kb, IL-6, TNF-α, and IL-10 expression in control eyes (control [L]), MFD eyes (control [R]), 1% atropine-treated control eyes (atropine [L]), and 1% atropine-treated MFD eyes (atropine [R]). FIGS. 4B and 4F are immunohistochemical analysis of c-Fos, NF-Kb, IL-6, TNF-α, and IL-10 expression in control eyes (control [L]), MFD eyes (control [R]), 3% CSA-treated control eyes (CSA [L]), and 3% CSA-treated MFD eyes (CSA [R]). FIG. 4C illustrates the expression levels of inflammation-related transcription factors c-Fos and NF-κB following treatment with LPS or PGN, wherein “L” means control and “R” means MFD eyes. FIG. 4E illustrates the expression of inflammatory and anti-inflammatory cytokines TNF-α and IL-10 upon treatment with LPS and PGN, wherein “L” means control and “R” means MFD eyes.

FIG. 5A shows that MFD was induced in guinea pigs by covering the right eye with a cloth attached to the skin at a distance of at least 1 cm from the eye. FIGS. 5B and 5C are immunohistochemical analysis of MMP2, COL1, TGF-β, c-Fos and IL-10 expression in control eyes (L) and MFD eyes (R) treated with 1% atropine or left untreated.

FIGS. 6A and 6B show that primary scleral fibroblast cells were treated with or without 100 μM atropine for 24 hours. Immunofluorescence analyses were used to determine MMP2 and COL1 expression in primary scleral fibroblasts; nuclei were stained with 4′,6-diamidino-2-phenylindole (DAPI). FIGS. 6C and 6D are gelatin zymography of pro- and active MMP2 in scleral fibroblasts and ARPE-19 retinal pigment epithelial cells treated with 1 μg/mL of LPS with or without atropine. The MMP2 inhibitor diacerein was used as the control. FIG. 6E illustrates the effect of atropine on LPS-induced activation of NF-κB and AP1 through inhibition of the PI3K-AKT and MAPK pathways. ARPE-19 cells were treated with PBS (control), 100 ng/mL of LPS, or LPS+100 μM atropine for 30 minutes and then harvested for western blot analysis to determine the phosphorylation status of ERK (Thr202/Tyr204), AKT (Ser473), PI3K (p85(Tyr458)/p55(Tyr199)), NF-κB (p65, Ser536), and c-Fos (Ser32).

Cumulative incidence of myopia is shown for control subjects and patients with systemic lupus erythromatosus (FIG. 7A), Kawasaki disease (FIG. 7B), and type 1 diabetes mellitus (FIG. 7C).

FIG. 8 illustrates the effect of suppressing inflammation in control eyes (left eye) and MFD eyes (right eyes) on myopia progression by various concentrations of resveratrol (0 mM, 3 mM, 30 mM, 100 mM).

FIG. 9 is an immunohistochemical analysis of COL1, TGF-β, and TNF-α expression in control eyes (left eyes) and MFD eyes (right eyes) treated with 100 mM resveratrol or untreated.

FIG. 10 illustrates the effect of resveratrol on the expression of LPS-induced monocyte chemotactic protein-1 (MCP-1).

FIG. 11 illustrates the result of resveratrol to LPS stimulation pathway, related to phosporylated-Akt and Akt via Western blotting and quantized data.

FIG. 12 illustrates the effect of resveratrol on LPS stimulation pathway, related to phosporylated-ERK1/2 and ERK1/2 via Western blotting and quantized data.

FIG. 13 is a schematic diagram, showing the pharmaceutical composition of the present invention for inhibiting the progression of myopia, wherein positive signs indicate promoting process; wherein minus signs indicate the suppression process.

### DETAILED DESCRIPTION OF THE PREFERRED EMBODIMENTS

Other objectives, advantages and novel features of the invention will become more apparent from the following detailed description when taken in conjunction with the accompanying drawings.

### Preparation Example 1

### Animal Assays

In this study a total of 160 golden Syrian hamsters aged 3 weeks, weighing 80 to 90 g and 20 albino guinea pigs 2 to 3 weeks of age were used for experiments. All animals were kept in a 12-hour light/dark cycle. All procedures were approved by the Institutional Animal Care and Use Committee of China Medical University and were conducted in accordance with the guidelines of the Use of Animals in Ophthalmic and Vision Research. Hamsters were raised with right eyelid fusion for 21 days. Myopia was induced in guinea pigs by covering the right eye with a cloth attached to the skin at a distance of at least 1 cm from the eye. MFD was induced in the right eye (with the left eye serving as a control) of animals randomly assigned to treatment or control groups (n=10 animals each) receiving daily applications of drug or phosphate-buffered saline (PBS), respectively, to both eyes.

### Preparation Example 2

### Cell Culture

R28 rat retinal epithelial cells were provided by Gail Seigel at the Ross Eye Institute (SUNY, Buffalo, N.Y., USA). The cells were cultured in Dulbecco's modified Eagle's medium (DMEM) with 10% fetal bovine serum (FBS) at 37° C. and 5% CO₂, with medium replacement every 3 days to 4 days. Sclera were placed in a 60-mm culture dish in DMEM supplemented with 10% FBS to isolate primary scleral fibroblasts; those from fewer than three passages were used in experiments. Cells were seeded in six-well plates (1×10⁵ cells/well) and treated with lipopolysaccharide (LPS) at 100 ng/mL or left untreated for 4 hours, followed by 100 μM atropine for 24 hours. Cell lysates were collected for quantitative (q)PCR to determine gene expression levels.

### Preparation Example 3

### cDNA Microarray Analysis

Sclera tissues were obtained from eyes with or without MFD. Total RNA was isolated using the RNeasy Mini Kit (purchased from Qiagen). RNA integrity and purity were determined with an Agilent Bioanalyser. A total of five unique total RNAs were pooled together (equal amounts) for cDNA microarray analysis. cDNA microarray analysis was performed using Affymetrix GeneChip Human Genome U133 Plus 2.0 and the procedures were consistent with the manufacturer's guidelines. cDNA microarrays were scanned using a GeneArray scanner. The image files (.cel format) were analyzed using the DNA-Chip Analyser software. Genes that exhibited a differential expression greater than 1.2 fold between the control and myopic eyes were selected for ingenuity pathway analysis.

### Preparation Example 4

### Physiological Measurements and Tissue Preparation

The RE (i.e., spherical-component RE, which is defined as the mean RE in horizontal and vertical meridians) was measured using a hand-held streak retinoscope. Animals were anesthetized with 10% ether in O₂. Ocular refraction was evaluated at the start and end of the experiment. At the end of the study, animals were sacrificed through CO₂ asphyxiation according to the guidelines of the Public Health Service, Office of Laboratory Animal Welfare, National Institutes of Health, and American Association of Veterinary Medicine. Eyes were enucleated using a razor blade on an ice plate under a surgical microscope (Topcon, Tokyo, Japan) by cutting perpendicularly to the anterior-posterior axis approximately 1 mm posterior to the ora serrata. The iris and ciliary body of the anterior segment of the eye were separated. Posterior sclera was excised using a 7-mm-diameter trephine. The axial lengths were determined through A-scan ultrasonography (PacScan 300 Plus, NY, USA). The average of 10 unique measurements was used.

### Preparation Example 5

### Gene Expression Profiling Using a PCR Array

Total RNA of sclera tissues were isolated using an RNeasy Mini Kit (Qiagen) and preceded for PCR array analysis. RNA integrity and purity were determined using an Agilent Bioanalyser. One microgram of total RNA in a final volume of 20-μL was reverse transcribed using the High-Capacity cDNA Reverse Transcription Kit (Applied Biosystems). The expression of genes involved in myopia progression was determined using a 96-well RT2 Profiler PCR Arrays-Human Autophagy (Qiagen, Frederick, Md., USA) in a LightCycler 480 PCR system (Roche, Germany).

### Preparation Example 6

### Immunofluorescence Staining

Primary sclera fibroblast cells plating on cover slides were washed with Tris-buffered saline (TBS), and subsequently fixed with 4% paraformaldehyde and washed twice with TBS before blocking with 1% BSA and 0.1% Triton X-100 for 1 hour. The cells were incubated with anti-MMP2 or anti-COL1 for 1 hour before being washed three times with TBS and subsequently incubated with an appropriate secondary antibody and a 4′,6-diamidino-2-phenylindole (DAPI) DNA stain. After being washed three times with TBS, the cells were imaged using fluorescence microscopy. All experiments were performed at least in triplicate.

### Preparation Example 7

### Analysis of MMP2 and MMP9 Activities

To facilitate subsequent analysis, 1×10⁶ cells were seeded in 24-well plates for at least 12 hours. Cells were washed with PBS three times and incubated with a culture medium without FBS in the presence or absence of 100 ng/mL of LPS or 100 ng/mL of LPS+100 μM atropine or 100 ng/mL of LPS+50 μM diacerein. Culture supernatants were collected after 48 hours and mixed with an equal volume loading buffer (125 mM Tris-HCl, pH 6.8, 3% SDS, 40% glycerol, and 0.02% bromophenol blue). To measure the MMP-2/MMP-9 activities, samples were separated using 8% SDS-PAGE containing 0.1% gelatin.

### Preparation Example 8

### Western Blot Analysis

ARPE-19 human retinal pigment epithelial cells were obtained from the Bioresource Collection and Research Center, HsinChu, Taiwan (BCRC; BCRC-60383). Cells were cultured in DMEM with 10% FBS at 37° C. and 5% CO2, with medium replacement every 3 to 4 days. ARPE-19 cells were treated with PBS (control), 100 ng/mL of LPS (Sigma), or LPS+100 μM atropine for 30 minutes. After treatment, 30 μg of total cell lysates was subjected to sodium dodecyl sulfate polyacrylamide gel electrophoresis (SDS-PAGE), followed by immunoblot analysis. Primary antibodies used included ERK (Thr202/Tyr204), AKT (Ser473), PI3K (p85[Tyr458]/p55[Tyr199]), NF-κB (p65, Ser536), and c-Fos (Ser32; Cell Signaling, Beverly, Mass., USA). Antirabbit or antimouse secondary antibody conjugated with horseradish peroxidase was also used. Immunoreactive protein bands were detected using an enhanced chemiluminescence kit (ECL, Pierce, Thermo Fisher Scientific, Pittsburgh, Pa., USA). Equal loading was confirmed through probing the blots with β-actin antibody (Abcam, Cambridge, Mass., USA) as well as anti-ERK, AKT, PI3K, NF-κB, and c-Fos.

### Preparation Example 9

### qPCR

Total RNA was extracted using the RNeasy MiniKit (Qiagen, Valencia, Calif., USA), and 5 μg of RNA was reverse-transcribed to cDNA by using the Superscript First Strand Synthesis system (Invitrogen, Carlsbad, Calif., USA). Primers and probes used for qPCR were selected from the Universal Probes Library (Roche, West Sussex, UK). Transcript levels were normalized to those of glyceraldehyde 3-phosphate dehydrogenase in each sample.

### Preparation Example 10

### Immunohistochemistry

Eyes were collected from atropine-treated and control animals, embedded in paraffin, and cut at a thickness of 20 μm; subsequently, the sections were placed on glass slides. Antigen retrieval was performed by boiling the slides in citrate buffer (pH 6.0); the sections were then stained with antibodies against IL-6, TNF-α, TGF-β, MMP2, c-Fos, NF-κB, and CHRM1 and 3. The EnVision System peroxidase kit (DAKO, Carpentaria, Calif., USA) was used to visualize immunoreactivity.

### Preparation Example 11

### Nationwide Population-Based Retrospective Cohort Study

Data source The NHIRD, maintained by the National Health Research Institute, is population-based and derived from the claims data of the National Health Insurance program, a mandatory-enrollment, single-payment system created in 1995, now covering over 99% of Taiwan's population. The database contains all medical claims and the information of insurants and provided a valuable resource, unique opportunity, and sufficiently large sample size for this study. The high validity of the diagnostic data from the NHIRD has been previously reported. Files for children (age<18 y) included 50% of those randomly selected from the Children's Registry from 1996 to 2008. To ensure the accuracy and reliability of the diagnoses, the index of inflammatory diseases, including SLE, T1DM, and Kawasaki disease (KD), was coded based on the International Classification of Diseases, Ninth Revision, Clinical Modification (ICD-9-CM) and the Registry for Catastrophic Illness Patient Database (published by the Department of Health, Executive Yuan, Taiwan), which includes selected major injuries or illnesses. The degree of urbanization was divided into seven categories based on a previous report, with Levels 1 and 7 representing the highest and lowest degrees, respectively. Because there were few children in Levels 5 to 7, these were combined with Level 4. Because of the personal electronic data privacy regulation, insurants' identities are encrypted before data are released to researchers. This study was approved by the Institutional Review Board of China Medical University Hospital.

Study sample Children newly diagnosed with SLE (ICD-9-CM code 710.0) between 2000 and 2004 formed the SLE cohort. The date of SLE diagnosis was the baseline. For each child with SLE, four non-SLE children were randomly selected who were frequency matched by sex, age (±1 y), urbanization level, parental occupation, and baseline year. Patients diagnosed with myopia (ICD-9-CM code 367.1) before the index date were excluded. The SLE and non-SLE cohorts were followed up until myopia appeared or were censored because of loss to follow-up, death, or for being otherwise unavailable before Dec. 31, 2008 Similar cohort analyses for investigating the occurrence of myopia were performed in T1DM (ICD-9-CM codes 250.X1 and 250.X3) and KD (ICD-9-CM code 446.1) cohorts, each with an appropriate comparison cohort.

Statistical analysis patients and control groups were compared regarding the distribution of demographic factors, including sex, age, urbanization level, and parental occupation by performing an χ2 test. The incidence rate and hazard ratio of myopia were calculated for SLE versus non-SLE, T1DM versus non-T1DM, and KD versus non-KD cohorts by using Cox proportional hazards regression analysis.

### Preparation Example 12

### Preparation of Resveratrol Solution

0.1141 g resveratrol (purchased from Sigma Aldrich Co.) powder was dissolved in 6 ml of ethanol to obtain resveratrol liquid. 1.46 g β-cyclodextrin (purchased from Sigma Aldrich Co.) was dissolved in 2 ml of sterile water to obtain a β-cyclodextrin solution (as a co-solvent); resveratrol liquid and β cyclodextrin were mixed in mole number of 1:2 by slowly adding dropwise β cyclodextrin solution into resveratrol liquid to form a mixture. The mixture was frozen drained, and then 5 ml artificial tears were added (purchased from Alcon company) to be re-dissolved to obtain 100 mM resveratrol solution. Resveratrol solution can be diluted by fetal bovine serum medium or artificial tears for various embodiments.

### Preparation Example 13

### Preparation of Diacerein Solution

8 ml artificial tears (purchased from Alcon company) was added in a 15 ml test tube, then 100 μl tween 80 and 30 mg of castor oil were added into the test tube. 40 mg diacerein (purchased from Sigma-Aldrich) was dissolved by gradually dropping under oscillating condition. Finally, 10 ml artificial tears was added to enhance dissolution by oscillator for 30 minutes to obtain final concentration 10 mM diacerein.

### Preparation Example 14

### Preparation of Diclofenac Solution

30 mg diclofenac sodium was added to 5 ml artificial tears and then fully dissolved to obtain a final concentration 6 mg/ml (0.6 w/v %) diclofenac solution by oscillator

15 mg diclofenac sodium was added to 5 ml artificial tears and then fully dissolved to obtain a final concentration 3 mg/ml (0.3 w/v %) diclofenac solution by oscillator

### Example 1

### Myopia Progression is Inhibited Through Atropine Treatment

According to the MFD animal model of Preparation example 1 and Physiological measurements of Preparation example 4, the effect of atropine for treating myopia progression can be displayed as in Table 1.

TABLE 1
Refractive power before and after atropine administration in hamsters
with MFD animal model

| | | Atropine | | Atropine | | Atropine | |
|---|---|---|---|---|---|---|---|
| | Control | (0.125%) | P value^{#} | (0.5%) | P value^{#} | (1%) | P value^{#} |
| BR | 11.92 ± 0.19 | 11.9 ± 0.07 | | 11.61 ± 0.1 | | 11.79 ± 0.15 | |
| BL | 11.83 ± 0.19 | 11.85 ± 0.18 | | 11.69 ± 0.13 | | 11.85 ± 0.18 | |
| FR | 4.65 ± 0.37 | 6.45 ± 0.1 | <0.0001 | 6.72 ± 0.11 | <0.0001 | 7.06 ± 0.29 | <0.0001 |
| FL | 7.68 ± 0.34 | 8.32 ± 0.16 | 0.0052 | 8.58 ± 0.12 | 0.005 | 10.79 ± 0.16 | <0.0001 |
| FR − BR | −7.22 ± 0.22 | −5.45 ± 0.07 | <0.0001 | −4.89 ± 0.18 | <0.0001 | −4.74 ± 0.39 | <0.0001 |
| FL − BL | −4.17 ± 0.49 | −3.53 ± 0.28 | <0.0001 | −3.11 ± 0.19 | <0.0001 | −1.06 ± 0.22 | <0.0001 |

BR: right eye before MFD;
BL: left eye before MFD;
FR: right eye after MFD (occluded eye);
FL: left eye after MFD.
^{#}Student's t test for paired comparisons between control and atropine-treated groups.
P < 0.05 was considered statistically significant.

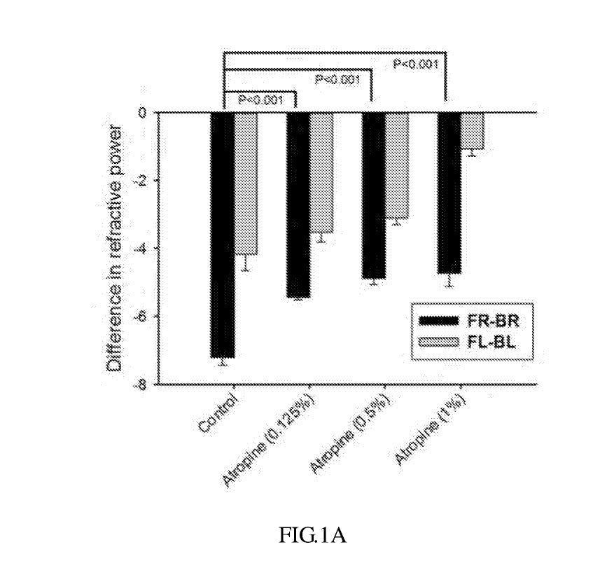

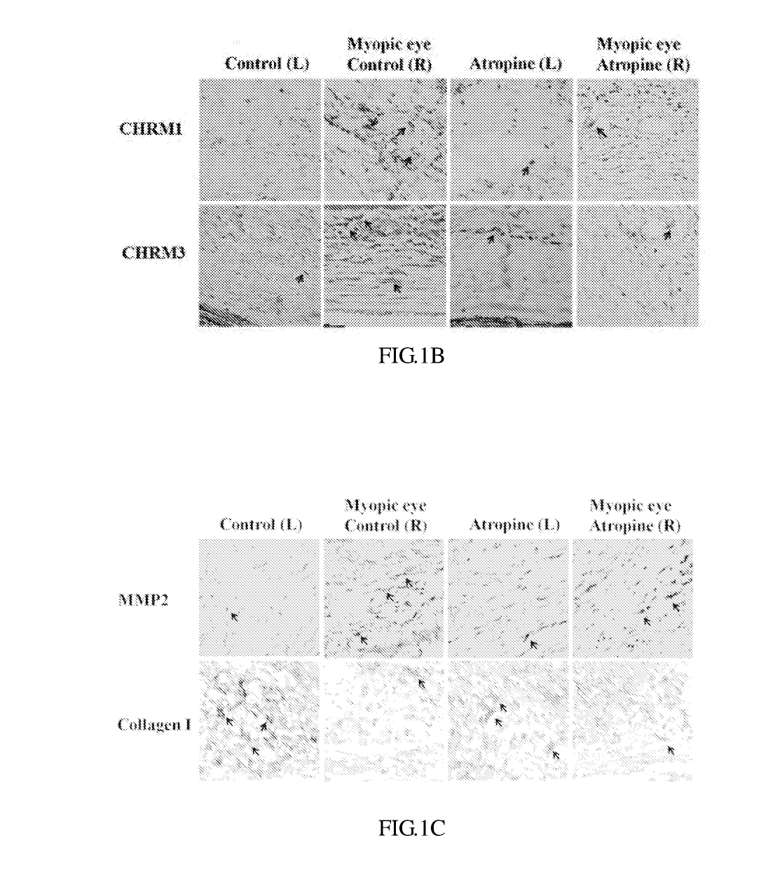

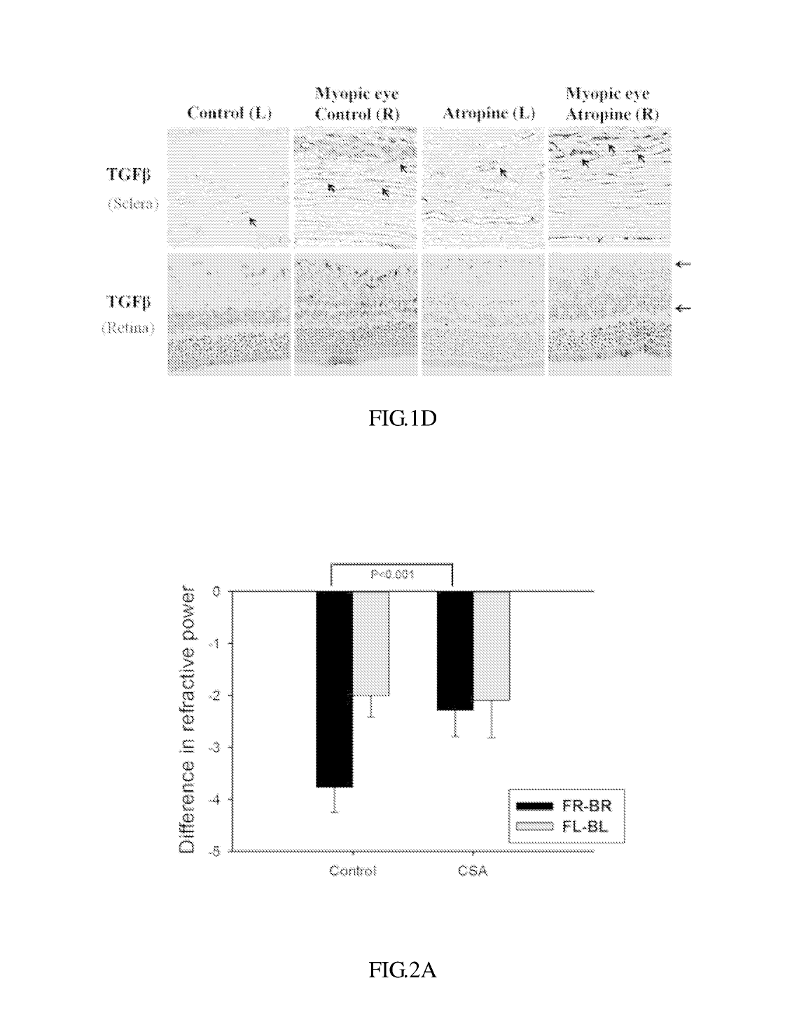

As shown in Table 1, the monocular deprivation animal model was used to study the relationship between inflammation and myopia. No difference was observed in refractive power between right and left eyes before MFD. As shown in Table 1 and FIG. 1, after 21 days, the REs for the PBS-treated MFD group were 4.65±0.37 and 7.68±0.34 D for MFD (right) and non-MFD (left) eyes, respectively (P<0.0001). The RE increased for both eyes as a function of atropine concentration: at 0.125%, 0.5%, and 1% atropine, the RE values were 6.45±0.1 D, 6.72±0.11 D, and 7.06±0.29 D, respectively, for the MFD eye, and 8.32±0.16 D, 8.58±0.12 D, and 10.79±0.16 D, respectively, for the non-MFD eye. These data suggest that atropine administration inhibits myopia progression.

### Example 2

### Genes Involved in Tissue Remodeling are Upregulated in Myopia

To affirm the induction of myopia in our animal model, we determined the expression levels of TGF-β and MMP2 in the sclera by using quantitative real-time PCR.

TABLE 2
Gene expression levels in scleral tissue and fibroblasts and retinal epithelial
cells as determined by real-time PCR

| | | Sclera | | Retinal cell (R28) | | Sclera fibroblast | |
|---|---|---|---|---|---|---|---|
| Gene Symbol* | Accession No. | CL** | CR** | LPS*** | LPS-Atropine**** | LPS*** | LPS-Atropine**** |
| Chrm 1 | NM_080773.1 | 1 ± 0.17 | 1.54 ± 0.12# | 1.39 ± 0.13 | 0.65 ± 0.16# | 1.49 ± 0.18 | 0.73 ± 0.11# |
| Chrm 2 | NM_031016.1 | 1 ± 0.14 | 0.98 ± 0.15# | 0.45 ± 0.23 | 1.96 ± 0.2# | 1.48 ± 0.17 | 1.24 ± 0.14 |
| Chrm 3 | NM_012527.1 | 1 ± 0.15 | 1.68 ± 0.21# | 1.27 ± 0.2 | 0.78 ± 0.11# | 1.58 ± 0.15 | 0.70 ± 0.23# |
| Chrm 4 | NM_031547.1 | 1 ± 0.2 | 0.89 ± 0.3 | 0.94 ± 0.15 | 0.81 ± 0.23 | 1.07 ± 0.23 | 1.11 ± 0.17 |
| Chrm5 | NM_017362.4 | 1 ± 0.22 | 0.84 ± 0.14 | 0.75 ± 0.21 | 0.78 ± 0.24 | 0.93 ± 0.15 | 0.98 ± 0.12 |
| Fos | DQ089699.1 | 1 ± 0.1 | 1.25 ± 0.18# | 1.39 ± 0.11 | 0.76 ± 0.17# | 1.00 ± 0.11 | 0.93 ± 0.17 |
| IL10 | NM_012854.2 | 1 ± 0.13 | 0.58 ± 0.1# | 0.56 ± 0.19 | 1.81 ± 0.15# | 0.56 ± 0.19 | 1.05 ± 0.21# |
| IL-6 | NM_012589.1 | 1 ± 0.18 | 2.05 ± 0.26# | 2.63 ± 0.15 | 0.86 ± 0.19# | 3.02 ± 0.16 | 0.79 ± 0.18# |
| IL1b | NM_031512.2 | 1 ± 0.12 | 1.87 ± 0.14# | 4.3 ± 0.25 | 0.17 ± 0.24# | 2.84 ± 0.21 | 0.75 ± 0.13# |
| Tgfb1 | NM_021578.2 | 1 ± 0.13 | 1.49 ± 0.12# | 1.18 ± 0.12 | 0.82 ± 0.11# | 2.15 ± 0.23 | 0.48 ± 0.24# |
| Tnfa | AF269159.1 | 1 ± 0.15 | 1.54 ± 0.15# | 1.56 ± 0.17 | 0.84 ± 0.24# | 1.49 ± 0.15 | 0.80 ± 0.2# |
| NFkB | AF079314.1 | 1 ± 0.1 | 1.52 ± 0.14# | 1.13 ± 0.11 | 0.85 ± 0.08# | 4.50 ± 0.27 | 0.36 ± 0.3# |
| MMP2 | NM_031054.2 | 1 ± 0.19 | 1.59 ± 0.08# | 1.32 ± 0.13 | 0.75 ± 0.16# | 1.79 ± 0.17 | 0.74 ± 0.19# |

*Genes that were differentially expressed between CR and CL in the cDNA microarray.
**CR: form-deprived right eye (occluded eye); CL: left eye.
***Cells were treated with 100 ng/ml LPS for 4 hours.
****Cells were treated with 100 ng/ml LPS and 100 μM atropine for 4 hours.
#Statistically significant (P < 0.05) by the Student's t test for paired comparisons between LPS and LPS+atropine

As shown in table 2, the expression levels of TGF-β and MMP2 were higher by 1.49- and 1.59-fold, respectively, in MFD eyes (P<0.05; Table 2). Whereas the expressions of CHRM2, CHRM 4, and CHRM 5 were similar between groups, the CHRM1 and CHRM3 levels were 1.54- and 1.68-fold higher, respectively, in MFD than they were in non-MFD eyes (P<0.05).

As shown in FIG. 1B, CHRM1 and CHRM 3 expression levels were higher in the sclera of the MFD than in the non-MFD eyes; CHRM1 and CHRM 3 were downregulated in MFD eyes after atropine treatment compared with the levels in PBS-treated MFD eyes. As shown in FIGS. 1C and 1D, treatment with 1% atropine decreased the MMP2 and increased COL1 expression in the sclera of the MFD eye compared with the results obtained for PBS treatment, which was confirmed through immunohistochemistry. Furthermore, the TGF-β level was upregulated by MFD in both the retina and sclera, but this effect was suppressed by atropine. These results indicate that the expression of genes that promote myopia progression through tissue remodeling was altered by MFD but corrected by atropine treatment.

### Example 3

### Genes Involved in Inflammation are Upregulated in Myopia

According to the microarrays of Preparation example 3 and the method of Preparation example 5, over 200 genes differentially expressions were identified in the sclera of PBS-treated MFD and non-MFD eyes expressed through microarray analysis. After ingenuity pathway analysis as Table 2 showed, c-fos and nuclear factor kappa b (NF-κB), two major transcription factors in regulating inflammatory reaction, were overexpressed in MFD eyes. An inflammatory cytokine and receptor PCR array was used to determine the differential expression of genes in the sclera of MFD versus non-MFD eyes. The increases in transcript levels for the transcription factors c-Fos and NF-κB were 1.25- and 1.52-fold higher, respectively, in the sclera of MFD than in non-MFD eyes (P<0.05. Other various inflammatory cytokines including interleukin-6 (IL-6) was 2.05-fold, TNF-α was 1.54-fold, TGF-β was 1.49-fold, and IL-1β was 1.87-fold (P<0.05). By contrast, the expression of the anti-inflammatory cytokine IL-10 was 0.58-fold lower in MFD than in non-MFD eyes (P<0.05). Since atropine affects both the sclera and retina, the expression of differentially expressed genes identified through the microarray was examined in rat R28 retinal cells and hamster primary scleral fibroblasts in which inflammation was induced by LPS. The expression of CHRM1 and 3, c-Fos, IL-6 and -1β, TGF-β, TNF-α, and NF-κB was upregulated by LPS treatment, but the effect was suppressed in both cell types in the presence of atropine (P<0.05). By contrast, IL-10 expression was suppressed by LPS and enhanced by atropine (P<0.05). These results suggest that the inflammatory response is linked to myopia progression.

### Example 4

### Suppressing Inflammation Inhibits the Progression of Myopia

To determine whether decreased inflammation inhibits myopia progression, the immunosuppressive agent cyclosporine A (CSA) was applied to the eyes of hamsters and the RE was measured on Day 21.

TABLE 3
Refractive power of monocular form deprivation (MFD) Syrian
hamster before and after cyclosporine A (CSA) administration

| | Control | CSA | P value^{#} |
|---|---|---|---|
| BR | 11.68 ± 0.37 | 11.57 ± 0.20 | |
| BL | 12.15 ± 0.63 | 12.00 ± 0.54 | |
| FR | 7.92 ± 0.54 | 9.25 ± 0.63 | 0.0071 |
| FL | 10.15 ± 0.25 | 9.90 ± 0.53 | >0.05 |
| FR-BR | −3.77 ± 0.48 | −2.29 ± 0.5 | 0.0003 |
| FL-BL | −2.00 ± 0.42 | −2.1 ± 0.72 | >0.05 |

BR: right eye before MFD;
BL: left eye before MFD;
FR: right eye after MFD (occluded eye);
FL: left eye after MFD.
^{#}student t test for paired comparison between control and CSA.
P < 0.05 is considered statistically significant.

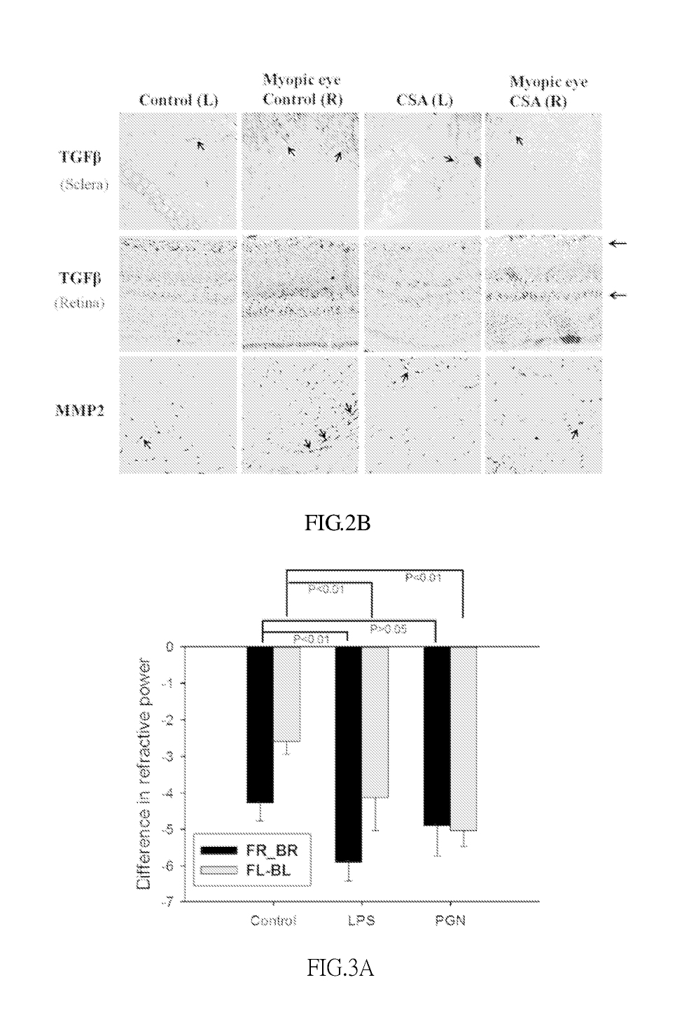

As shown in Table 3 and FIG. 2A, the REs for the PBS-treated group were 7.92±0.54 D and 10.15±0.25 D for the MFD and non-MFD eyes, respectively (P<0.0001). These values changed on treatment with 3% CSA to 9.25±0.63 D and 9.90±0.53 D, respectively, indicating that the progression of myopia was blocked. As shown in FIG. 2b, this was underscored by the concomitant decreases in MMP2 and TGF-β expression that were observed.

To test whether increased inflammation enhanced the progression of myopia, LPS and 500 ng/mL peptidoglycan (PGN), inducers of inflammation originating from Gram-negative and -positive bacterial cell walls, respectively, were applied to the eyes of MFD mice every second day for 21 days.

TABLE 4
Refractive power of monocular form deprivation (MFD) Syrian
hamster before and after lipopolysaccharide (LPS) and
peptidoglycan (PGN) administration.

| | Control | LPS | P value^{#} | PGN | P value^{#} |
|---|---|---|---|---|---|
| BR | 11.94 ± 0.49 | 12.30 ± 0.48 | | 11.48 ± 0.49 | |
| BL | 11.83 ± 0.6 | 11.92 ± 0.56 | | 11.73 ± 0.25 | |
| FR | 7.67 ± 0.74 | 6.44 ± 0.18 | 0.0069 | 6.47 ± 0.39 | 0.0125 |
| FL | 9.25 ± 0.48 | 7.79 ± 0.88 | 0.0116 | 6.78 ± 0.63 | 0.0001 |
| FR-BR | −4.27 ± 0.49 | −5.9 ± 0.54 | 0.0011 | −4.90 ± 0.83 | >0.05 |
| FL-BL | −2.58 ± 0.36 | −4.13 ± 0.9 | 0.0072 | −5.03 ± 0.43 | <0.0001 |

BR: right eye before MFD;
BL: left eye before MFD;
FR: right eye after MFD (occluded eye);
FL: left eye after MFD.
^{#}student t test for paired comparison between control and LPS or PGN.
P < 0.05 is considered statistically significant.

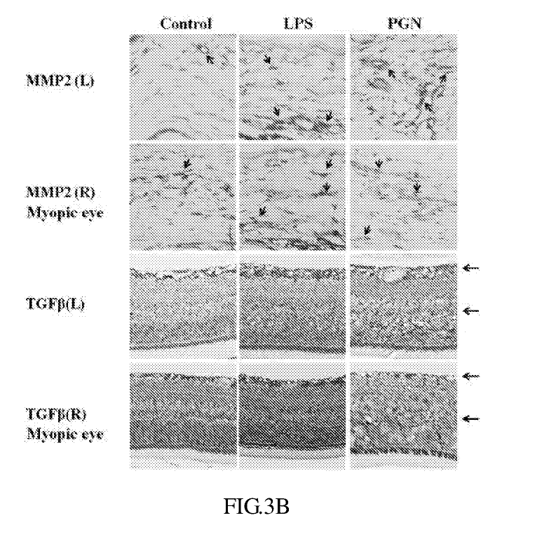

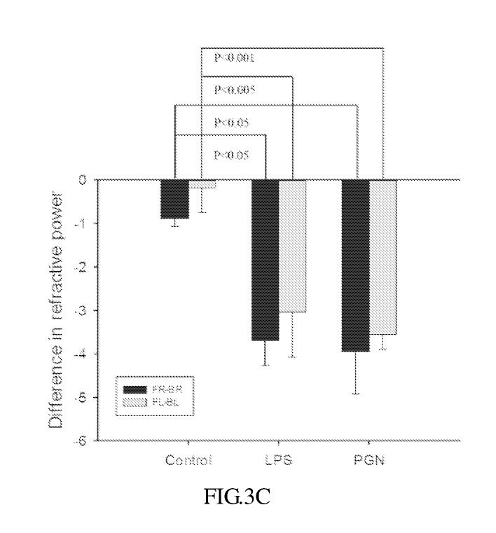

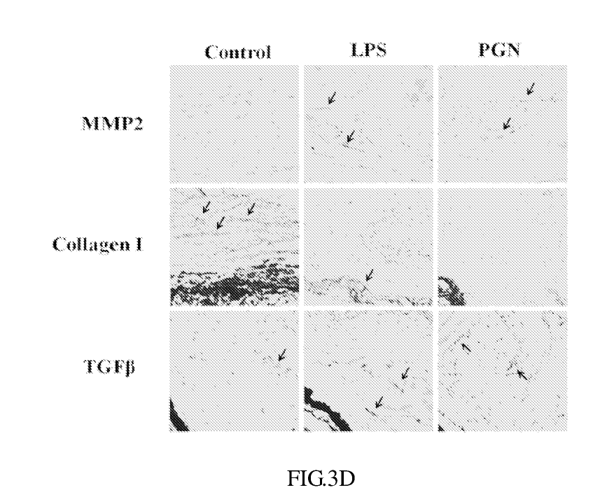

As shown in Table 4 and FIG. 3A, the RE for PBS-treated animals was 7.67±0.74 D and 9.25±0.48 D for MFD and non-MFD eyes, respectively (P<0.0001); however, these values decreased to 6.44±0.18 D and 7.79±0.88 D, respectively, on LPS treatment, and to 6.47±0.39 D and 6.78±0.63 D, respectively, on PGN treatment. As shown in FIG. 3B, the decrease in RE was accompanied by an upregulation of MMP2 and TGF-β.

As shown in FIG. 3A, myopia was induced in the non-MFD eye through LPS and PGN but not through PBS treatment (P<0.01), suggesting a direct link between inflammation and myopia progression. To further test this possibility, LPS and PGN were applied to the eyes of hamsters without MFD.

TABLE 5
Refractive power of Syrian hamster before and after
lipopolysaccharide (LPS) and peptidoglycan (PGN) administration.

| | Control | LPS | P value^{#} | PGN | P value^{#} |
|---|---|---|---|---|---|
| BR | 11.63 ± 0.53 | 12.25 ± 0.57 | | 12.17 ± 1.04 | |
| BL | 11.33 ± 1.18 | 12.28 ± 0.97 | | 12.22 ± 0.35 | |
| FR | 12.5 ± 0.18 | 8.56 ± 0.42 | 0.0001 | 9.14 ± 1.21 | 0.0089 |
| FL | 12.21 ± 0.29 | 8.33 ± 0.96 | 0.0026 | 8.67 ± 0.63 | 0.0009 |
| FR − BR | −0.88 ± 0.18 | −3.69 ± 0.57 | 0.0012 | −3.03 ± 1.04 | 0.0243 |
| FL − BL | −0.18 ± 0.57 | −3.95 ± 0.97 | 0.0044 | −3.55 ± 0.35 | 0.0009 |

BR: right eye before treatment;
BL: left eye before treatment;
FR: right eye after treatment (occluded eye);
FL: left eye after treatment.
^{#}student t test for paired comparison between control and LPS or PGN.
P < 0.05 is considered statistically significant.

As shown in Table 5 and FIG. 3C, after 21 days, the RE for the PBS-treated group was 12.5±0.18 D and 12.21±0.29 D for right and left eyes, respectively. These values decreased to 8.56±0.42 D and 8.33±0.96 D, respectively, on LPS treatment, and 9.14±1.21 D and 8.67±0.63 D, respectively, in PGN-treated eyes, representing statistically significant differences with respect to the PBS-treated group (P<0.001). As shown in FIG. 3D, these changes occurred concurrently with the upregulation of TGF-β and MMP2 expression.

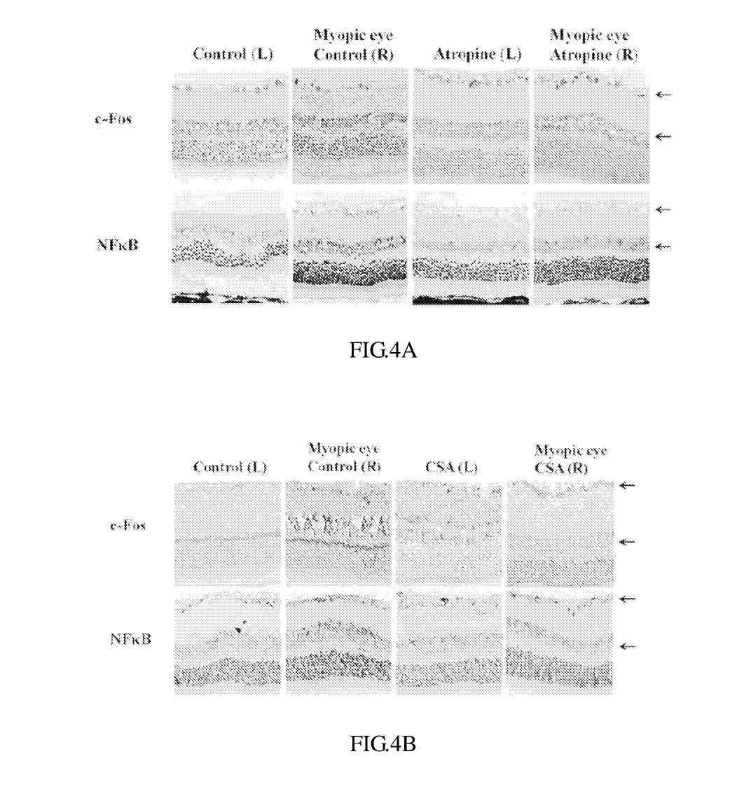

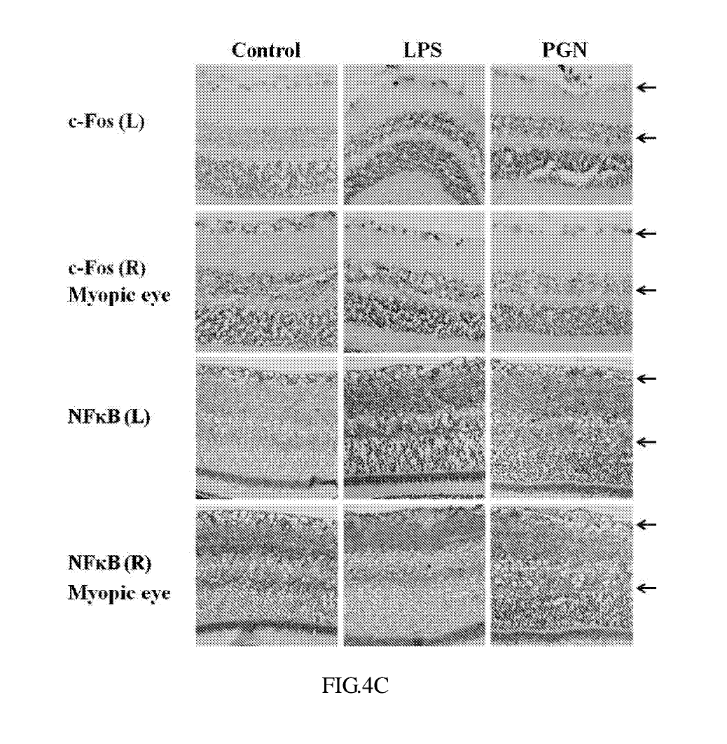

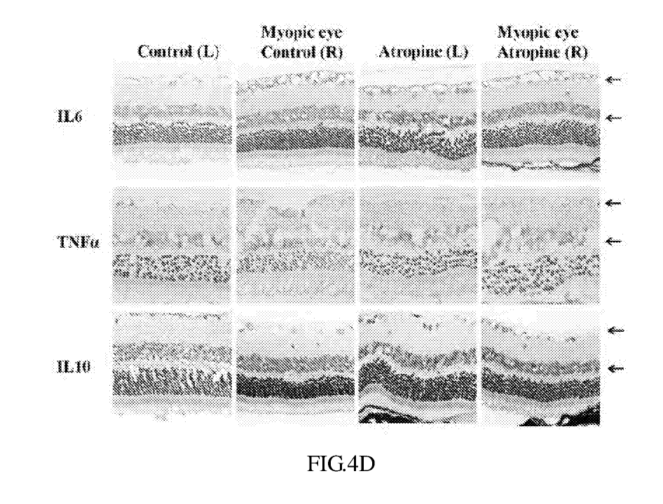

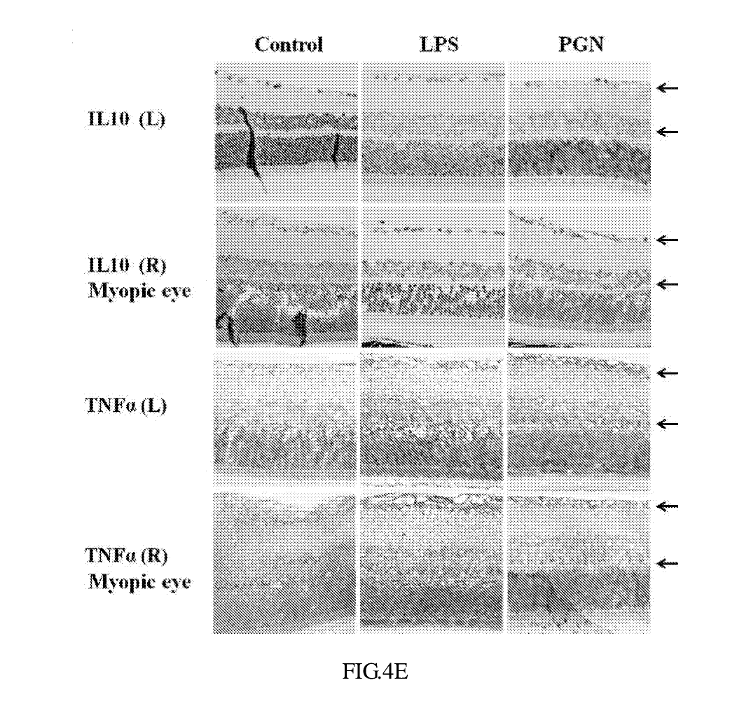

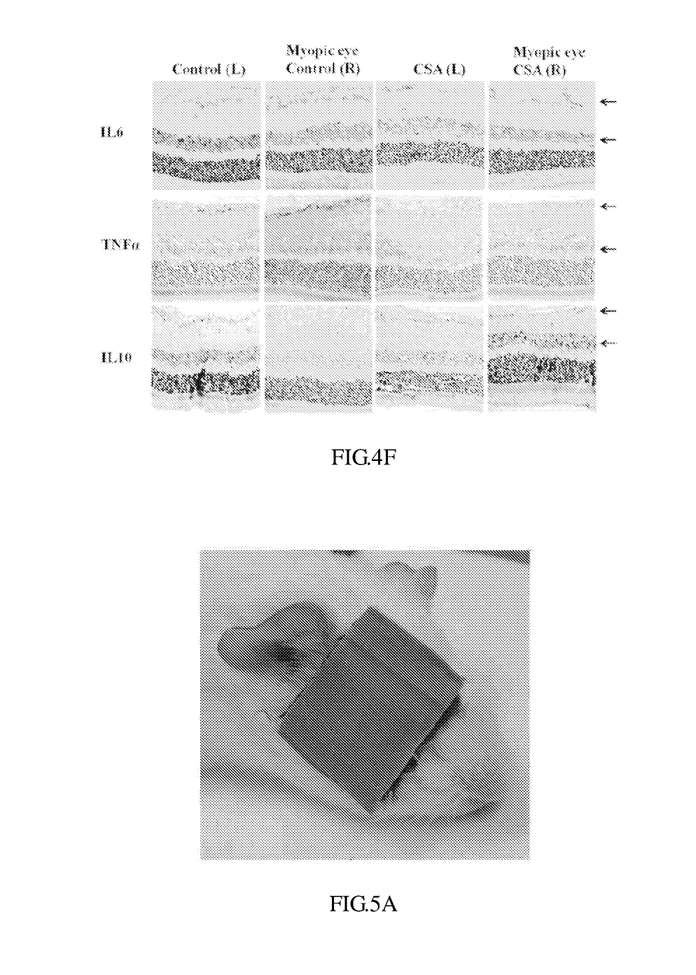

The expression of inflammatory molecules was evaluated through immunohistochemistry. As shown in FIG. 4A, the expression of c-Fos and NF-κB in the retinas of MFD eyes was higher than that in non-MFD eyes but was suppressed through treatment with 1% atropine. As shown in FIGS. 4B and 4C, CSA lowered the c-Fos and NF-κB expression that was stimulated by LPS or PGN. As shown in FIG. 4D, IL-6 and TNF-α immunoreactivity in the retina was elevated in MFD eyes, but the expression of these factors was reduced through application of 1% atropine. By contrast, the IL-10 expression was elevated following atropine treatment. As shown in FIG. 4E, IL-10 and TNF-α levels, which were increased through LPS or PGN treatment. As shown in FIG. 4F, IL-6 and TNF-α levels were lowered by CSA, but IL-10 level was increased after CSA treatment. Taken together, these results indicate that induced inflammation caused acceleration of myopia, and this acceleration could be reversed through application of anti-inflammatory agents.

We observed a similar increase in inflammatory response in hamsters as we did in a guinea pig model of MFD. As shown in FIG. 5A, MFD was induced in guinea pigs by covering the right eye with a cloth attached to the skin at a distance of at least 1 cm from the eye. 1% atropine was applied to the eyes of guinea pigs and the REs and axial lengths were measured on Day 21.

TABLE 3
The ocular components of guinea pig after atropine treatment

| | |
|---|---|
| Left eye (control eye) | Right eye (myopic eye) |

Refractive power (D)

| | | |
|---|---|---|
| Control | −1.08 ± 0.94 | −1.33 ± 0.82 |
| MFD | −0.42 ± 1.38 | −9.22 ± 0.93 |
| MFD + atropine | −1.50 ± 0.82 | −6.79 ± 1.00* |

Axial length (cm)

| | | |
|---|---|---|
| Control | 1.00 ± 0.00 | 1.03 ± 0.04 |
| MFD | 1.08 ± 0.00 | 1.17 ± 0.01 |
| MFD + atropine | 1.04 ± 0.82 | 1.14 ± 0.01* |

MFD: monocular form deprivation.
*Student t test for paired comparison between MFD and MFD + Atropine. A P < 0.05 is considered statistically significant. Values denoted with this symbol are all statistically significant.

As shown in Table 3, the REs for the PBS-treated group were −9.22±0.93 D and −0.42±1.38 D for MFD and non-MFD eyes, respectively. These values changed on treatment with atropine to −6.79±1.00 D and −1.50±0.82 D, respectively. The axial lengths for the PBS-treated group were 1.17±0.01 cm and 1.08±0.00 cm for MFD and non-MFD eyes, respectively. These values changed on treatment with atropine to 1.14±0.01 cm and 1.04±0.82 cm, respectively. Both REs and axial lengths exhibited statistical significance between PBS and atropine treated MFD eyes (all P<0.005). The expression levels of MMP2, TGF-β, and c-Fos increased in myopic eyes whereas that of COL1 decreased. As shown in FIGS. 5B and 5C, atropine treatment reduced MMP2, TGF-β, and c-Fos and increased COL1 expression in the sclera or retina of the MFD eye compared to the results obtained for PBS treatment. The IL-10 level was downregulated by MFD, but this effect was suppressed by atropine. These results revealed consistent outcomes when using various MFD methods in different animal species.

### Example 5

### Atropine Inhibits Phosphatidylinositol 3-Kinase (PI3K)-AKT-NF-κB and Extracellular Signal-Regulated Kinase (ERK)-Fos Pathways

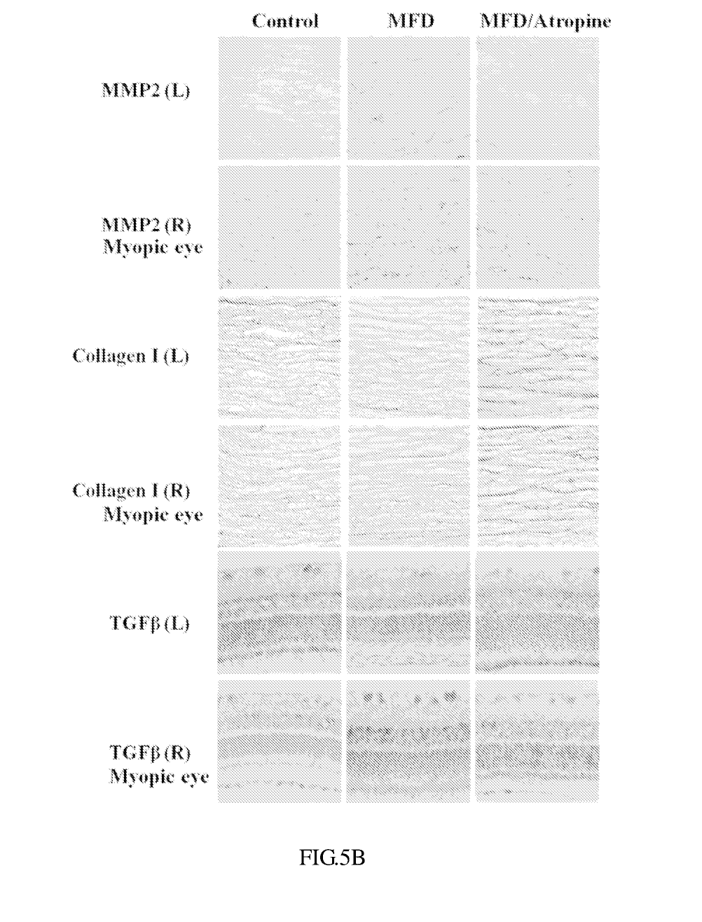

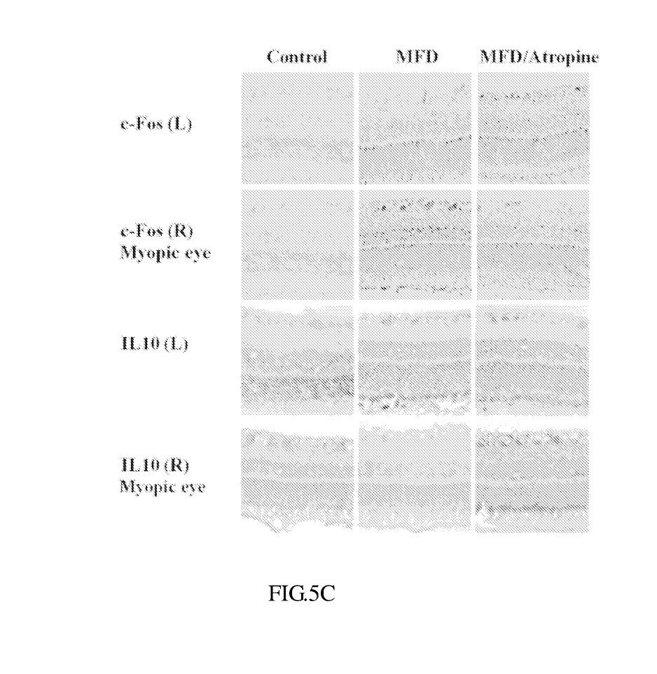

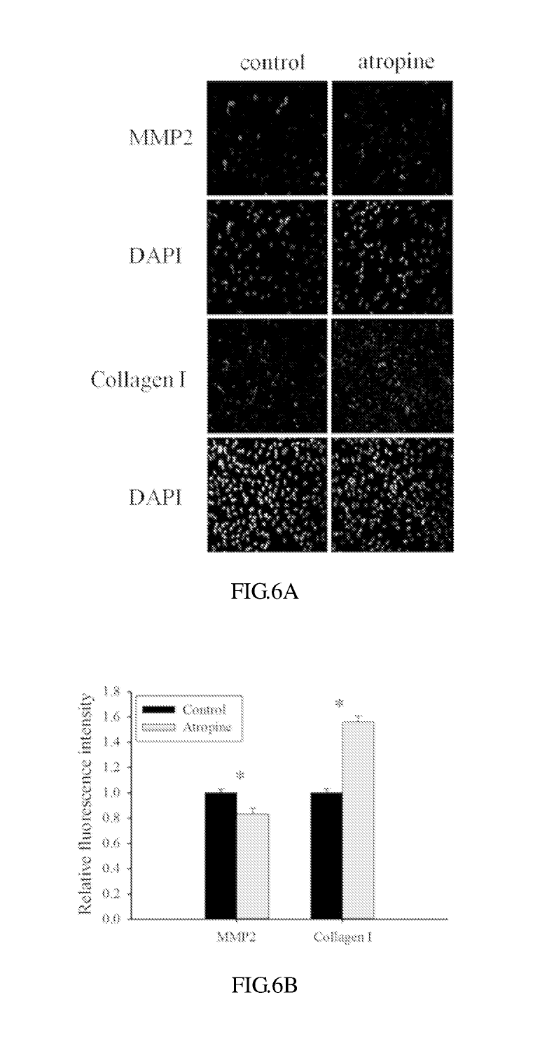

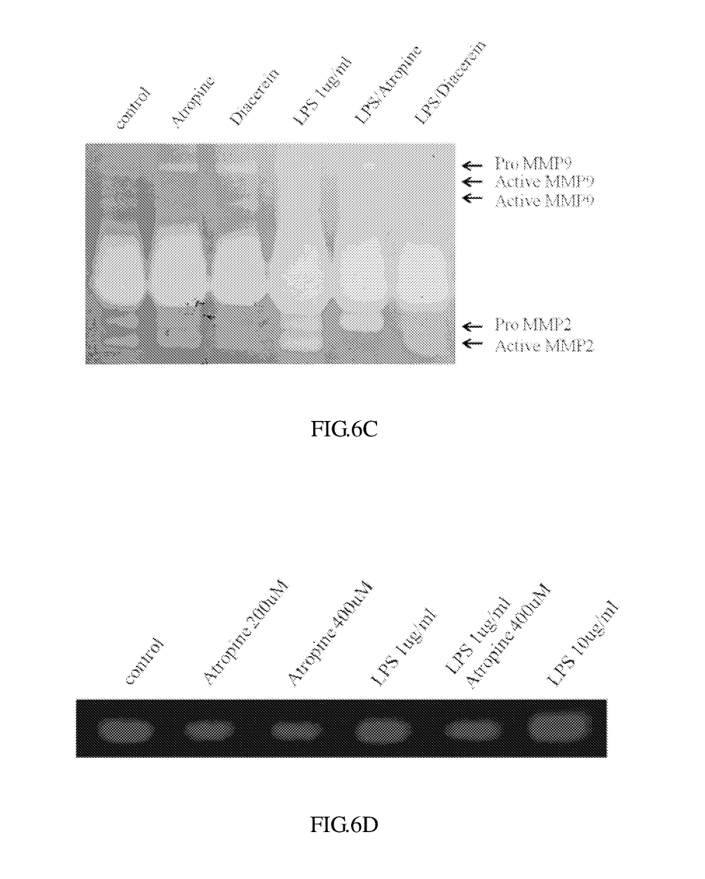

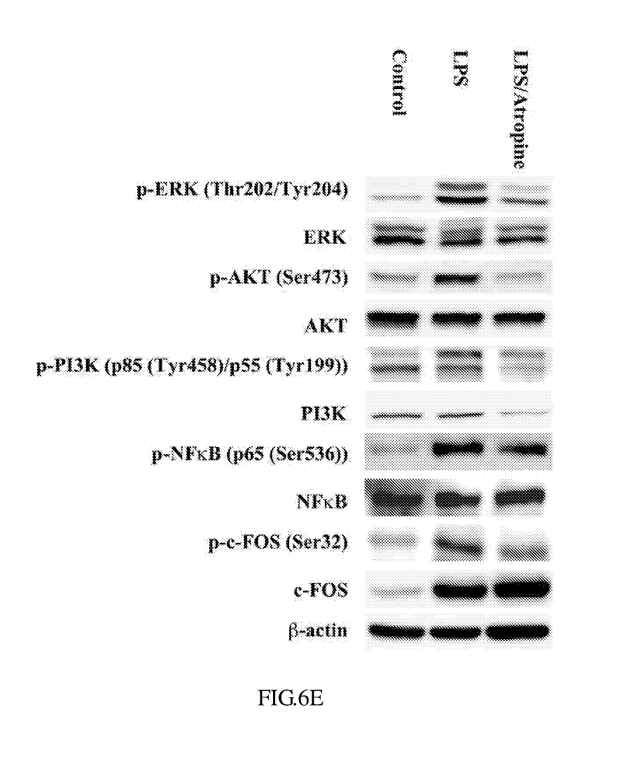

To determine the molecular mechanisms of atropine in inhibiting myopia progression, rat primary sclera fibroblast and human retinal pigment epithelial cells ARPE-19 were used. As shown in FIGS. 6A and 6B, atropine inhibited the expression levels of MMP2 and increased COL1 in primary sclera fibroblast. As shown in FIGS. 6C and 6D, MMP2 activities increased in cells activated by LPS whereas they decreased through application of atropine or diacerein. The results suggested that diacerein can be used as a new agent to inhibit myopia progression.

To study the signaling pathways influenced by atropine, human retinal pigment epithelial cells ARPE-19 were treated with LPS or LPS/atropine for 4 hours. As shown in FIG. 6E, the activation of ERK through LPS was inhibited by atropine treatment as well as its downstream signaling molecule c-FOS. Atropine treatment also inhibited the LPS activation of PI3K, AKT, and NF-κB. These results indicated that atropine inhibits inflammation through downregulation of ERK-c-FOS and PI3K-AKT-NF-κB pathways.

### Example 6

### Association Between Inflammatory Disorders and Subsequent Myopia Risk

According to the method of Preparation example 11, the retrospective cohort study was conducted using data on children (<18 years old) obtained from the National Health Insurance Research Database (NHIRD) to determine whether the inflammatory diseases SLE, KD, and T1D are associated with the incidence of myopia.

TABLE 7
Demographics between children with or without
systemic lupus erythematosus (SLE) status

| | non-SLE | | SLE | | |
|---|---|---|---|---|---|
| | n = 4856 | | n = 1214 | | |
| Variable | n | % | N | % | p-value |
| Gender | | | | | 0.99 |
| Girl | 3816 | 78.58 | 954 | 78.58 | |
| Boy | 1040 | 21.42 | 260 | 21.42 | |
| Age, years | | | | | 0.99 |
| 1-6 | 572 | 11.78 | 143 | 11.78 | |
| 7-12 | 1504 | 30.97 | 376 | 30.97 | |
| 13-18 | 2780 | 57.25 | 695 | 57.25 | |
| Means (SD) | 12.78 | (4.07) | 12.80 | (4.07) | 0.90 |
| Urbanization | | | | | 0.80 |
| Level 1 (highest) | 1360 | 28.16 | 335 | 27.85 | |
| Level 2 | 1526 | 31.59 | 383 | 31.84 | |
| Level 3 | 906 | 18.76 | 214 | 17.79 | |
| Level 4 (lowest) | 1038 | 21.49 | 271 | 22.53 | |
| Parents' occupational | | | | | 0.28 |
| status | | | | | |
| White collar | 2696 | 55.52 | 682 | 56.18 | |
| Blue collar | 1679 | 34.58 | 430 | 35.42 | |
| Other | 481 | 9.91 | 102 | 8.40 | |

Abbreviation:
SLE, systemic lupus erythematosus;
SD, standard deviation
†The urbanization level was categorized by the population density of the residential area into 4 levels, with level 1 as the most urbanized and level 4 as the least urbanized.
‡Other occupations included primarily retired, unemployed, or low income populations.

TABLE 8
Demographics between children with or without Kawasaki disease
(KD)

| | non-KD | | KD | | |
|---|---|---|---|---|---|
| | n = 2184 | | n = 546 | | |
| Variable | n | % | N | % | p-value |
| Gender | | | | | 0.99 |
| Girl | 780 | 35.71 | 195 | 35.71 | |
| Boy | 1404 | 64.29 | 351 | 64.29 | |
| Age, years | | | | | 0.99 |
| <1 | 676 | 30.95 | 169 | 30.95 | |
| 1-3 | 1228 | 56.23 | 307 | 56.23 | |
| 4-6 | 280 | 12.82 | 70 | 12.82 | |
| Means (SD) | 2.10 | (1.59) | 20.11 | (1.55) | 0.89 |
| Urbanization | | | | | 0.08 |
| Level 1 (highest) | 588 | 27.03 | 161 | 30.09 | |
| Level 2 | 672 | 30.90 | 144 | 26.92 | |
| Level 3 | 409 | 18.80 | 117 | 21.87 | |
| Level 4 (lowest) | 506 | 23.26 | 113 | 21.12 | |
| Parents' occupational | | | | | 0.003 |
| status | | | | | |
| White collar | 1326 | 60.71 | 375 | 68.68 | |
| Blue collar | 548 | 25.09 | 110 | 20.15 | |
| Other | 310 | 14.19 | 61 | 11.17 | |

Abbreviation:
KD, Kawasaki disease;
SD, standard deviation
†The urbanization level was categorized by the population density of the residential area into 4 levels, with level 1 as the most urbanized and level 4 as the least urbanized.
‡Other occupations included primarily retired, unemployed, or low income populations.

TABLE 9
Demographics between children with or without
type 1 diabetes mellitus (T1DM)

| | non-T1DM | | T1DM | | |
|---|---|---|---|---|---|
| | n = 2236 | | n = 559 | | |
| Variable | n | % | N | % | p-value |
| Gender | | | | | 0.99 |
| Girl | 1208 | 54.03 | 302 | 54.03 | |
| Boy | 1028 | 45.97 | 257 | 45.97 | |
| Age, years | | | | | 0.99 |
| 1-6 | 644 | 28.80 | 161 | 28.80 | |
| 7-12 | 916 | 40.97 | 229 | 40.97 | |
| 13-18 | 676 | 30.23 | 169 | 30.23 | |
| Means (SD) | 9.97 | (4.63) | 9.95 | (4.63) | 0.94 |
| Urbanization | | | | | 0.40 |
| Level 1 (highest) | 626 | 28.12 | 143 | 25.77 | |
| Level 2 | 716 | 32.17 | 171 | 30.81 | |
| Level 3 | 406 | 18.24 | 115 | 20.72 | |
| Level 4 (lowest) | 478 | 21.47 | 126 | 22.70 | |
| Parents' occupational | | | | | 0.04 |
| status | | | | | |
| White collar | 1268 | 56.71 | 293 | 52.42 | |
| Blue collar | 727 | 32.51 | 186 | 33.27 | |
| Other | 241 | 10.78 | 80 | 14.31 | |

Abbreviation:
T1DM, type 1 diabetes;
SD, standard deviation
†The urbanization level was categorized by the population density of the residential area into 4 levels, with level 1 as the most urbanized and level 4 as the least urbanized.
‡Other occupations included primarily retired, unemployed, or low income populations.

As shown in Tables 7 to 9, from 2000 to 2004, 1214 SLE, 546 KD, and 559 T1D patients were newly diagnosed and randomly matched for age, sex, and index year with patients without SLE, KD, or T1D from the general population at a 1:4 ratio.

TABLE 10
Incidence rates for myopia in both cohorts and hazard ratio for myopia in systemic
lupus erythematosus (SLE) cohort compared to non-SLE cohort, stratified by demographic factors

| | Non-SLE | | | SLE | | | Crude | Adjusted |
|---|---|---|---|---|---|---|---|---|
| Variables | Case | Person-year | IR | Case | Person-year | IR | HR (95% CI) | HR (95% CI) |
| Overall | 534 | 17595.49 | 30.35 | 173 | 4120.45 | 41.99 | 1.38 (1.16-1.64)*** | 1.40 (1.18-1.66)*** |
| Gender | | | | | | | | |
| Girl | 398 | 13305.40 | 29.91 | 131 | 3102.62 | 42.22 | 1.41 (1.16-1.72)** | 1.43 (1.18-1.74)*** |
| Boy | 136 | 4290.09 | 31.70 | 42 | 1017.84 | 41.26 | 1.30 (0.92-1.83) | 1.32 (0.93-1.87) |
| Age, years | | | | | | | | |
| 1-6 | 188 | 3257.25 | 57.72 | 66 | 689.90 | 95.67 | 1.68 (1.27-2.22)*** | 1.70 (1.28-2.25)*** |
| 7-12 | 282 | 8206.35 | 34.36 | 85 | 1938.54 | 43.85 | 1.26 (0.99-1.60) | 1.24 (0.97-1.58) |
| 13-18 | 64 | 6131.89 | 10.44 | 22 | 1492.01 | 14.75 | 1.41 (0.87-2.28) | 1.41 (0.87-2.29) |

Abbreviation:
SLE, systemic lupus erythematosus;
IR, incidence density rates, per 1,000 person-years;
HR, hazard ratio;
CI, confidence interval
Adjusted HR: mutually adjusted for age, gender, urbanization, and parents' occupational status in Cox proportional hazards regression
**P < 0.01,
***P < 0.001

TABLE 11
Incidence rates for myopia in both cohorts and hazard ratio for myopia in Kawasaki disease
(KD) cohort compared to non-KD cohort, stratified by demographic factors

| | | | | | | | Compared to | |
|---|---|---|---|---|---|---|---|---|
| | Kawasaki disease | | | | | | non-Kawasaki disease | |
| | Non-KD | | | KD | | | Crude | Adjusted |
| Variables | Case | Person-year | IR | Case | Person-year | IR | HR (95% CI) | HR (95% CI) |
| Overall | 437 | 13062.95 | 33.45 | 142 | 3210.72 | 44.23 | 1.33 (1.10-1.61)** | 1.26 (1.04-1.53)* |
| Gender | | | | | | | | |
| Girl | 165 | 4656.22 | 35.44 | 60 | 1119.66 | 53.59 | 1.54 (1.15-2.07)* | 1.48 (1.10-2.01)* |
| Boy | 272 | 8406.74 | 32.36 | 82 | 2091.06 | 39.21 | 1.21 (0.95-1.55) | 1.13 (0.88-1.45) |
| Age, years | | | | | | | | |
| <1 | 99 | 4365.42 | 22.68 | 31 | 1073.33 | 28.88 | 1.29 (0.86-1.94) | 1.29 (0.85-1.94) |
| 1-3 | 252 | 7234.12 | 34.83 | 90 | 1760.03 | 51.14 | 1.49 (1.17-1.89)** | 1.44 (1.13-1.84)** |
| 4-6 | 86 | 1463.41 | 58.77 | 21 | 377.37 | 55.65 | 0.96 (0.60-1.55) | 0.82 (0.50-1.36) |

Abbreviation:
KD, Kawasaki disease;
IR, incidence density rates, per 1,000 person-years;
HR, hazard ratio;
CI, confidence interval
Adjusted HR: mutually adjusted for age, gender, urbanization, and parents' occupational status in Cox proportional hazards regression
*P < 0.05,
**P < 0.01

TABLE 12
Incidence rates for myopia in both cohorts and hazard ratio for myopia in type 1 diabetes mellitus
(T1DM) cohort compared to non-T1DM cohort, stratified by demographic factors

| | T1DM | | | | | | Compared to non-T1DM | |
|---|---|---|---|---|---|---|---|---|
| | Non-T1DM | | | T1DM | | | Crude | Adjusted |
| Variables | Case | Person-year | IR | Case | Person-year | IR | HR (95% CI) | HR (95% CI) |
| Overall | 368 | 10228.61 | 35.98 | 137 | 2363.65 | 57.96 | 1.60 (1.32-1.95)*** | 1.59 (1.31-1.94)*** |
| Gender | | | | | | | | |
| Girl | 219 | 5696.52 | 39.84 | 80 | 1262.48 | 63.37 | 1.58 (1.23-2.05)*** | 1.57 (1.21-2.04)** |
| Boy | 149 | 4732.09 | 31.49 | 57 | 1101.17 | 51.76 | 1.63 (1.20-2.22)** | 1.62 (1.19-2.20)** |
| Age, years | | | | | | | | |
| 1-6 | 179 | 3619.78 | 49.45 | 53 | 875.36 | 60.55 | 1.23 (0.90-1.67) | 1.25 (0.92-1.69) |
| 7-12 | 173 | 4833.60 | 35.79 | 74 | 1054.57 | 70.17 | 1.89 (1.44-2.48)*** | 1.93 (1.47-2.54)*** |
| 13-18 | 16 | 1775.23 | 9.01 | 10 | 433.72 | 23.06 | 2.54 (1.15-5.61)* | 2.16 (0.95-4.91) |

Abbreviation:
T1DM, type 1 diabetes mellitus;
IR, incidence density rates, per 1,000 person-years;
HR, hazard ratio;
CI, confidence interval
Adjusted HR: mutually adjusted for age, gender, urbanization, and parents' occupational status in Cox proportional hazards regression
*P < 0.05,
**P < 0.01,
***P < 0.001

As shown in Tables 10 to 12, cohorts were followed until the end of 2008 when the incidence of myopia was assessed. The risk of myopia was 1.40-fold (95% CI=1.18-1.66) higher in the SLE cohort, 1.26-fold (95% CI=1.04-1.53) higher in the KD cohort, and 1.59-fold (95% CI=1.31-1.94) higher in the TID cohort, compared with the controls. As shown in FIGS. 7A to 7C, the cumulative incidence of myopia by the end of the follow-up period was 3.5%, 11.6%, and 7.9% higher in the SLE, KD, and T1D groups, respectively, than in controls (P<0.001). These findings provide clinical evidence for the association between inflammatory diseases and the occurrence of myopia.

### Example 7

### Effect of Resveratrol to MFD-Induced Myopia in Hamster

The refractive powers of hamsters were measured before experiment by refractometer (in China Medical University Hospital Department of Ophthalmology). The hamsters were administered artificial tears (referred to as control group) or various concentrations (3 mM, 30 mM, 100 mM) of resveratrol (experimental group) for five hamsters each group. The right eyes of the hamsters of each group were stitched; after 21 days, the stitches were all removed and then the refractive powers of each hamster were measured.

Diopter

| | | right myopic eye |
|---|---|---|
| | left eye (mean ± SD) | (mean ± SD) |
| control | −0.04 ± 1.12 | −4.33 ± 0.35 |
| 3 mM resveratrol | −0.17 ± 0.24 | −2.42 ± 1.30 |
| 30 mM resveratrol | −0.63 ± 0.41 | −2.46 ± 0.65 |
| 100 mM resveratrol | −0.25 ± 1.53 | −1.13 ± 1.47 |

As shown in Table 13, the diopter of the right myopic eye of the control group has increased significantly; after treatment with various concentrations of resveratrol, the diopter was decreasing significantly, especially 100 mM resveratrol. As shown in FIG. 8, the diopter difference of left eye and right eye were very obvious. After resveratrol administration with various concentration, myopia can be inhibited, wherein 100 mM resveratrol was the most effective.

### Example 8

### Effect of Resveratrol for Inflammation-Related Protein Induced Myopia and Myopic Related Proteins Expression

The expression of myopia related proteins: collagen I and inflammation-related proteins, such as TGF-β and tumor necrosis factor-α (TNF-α) were observed in myopic eyes.

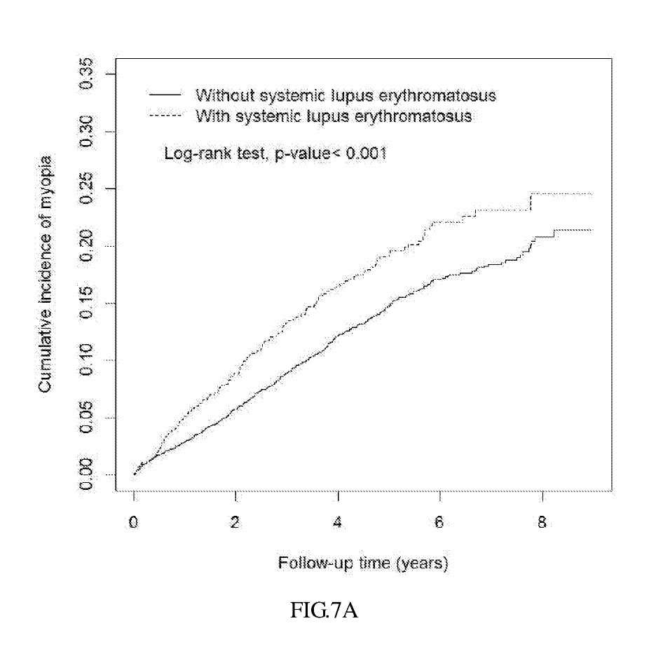

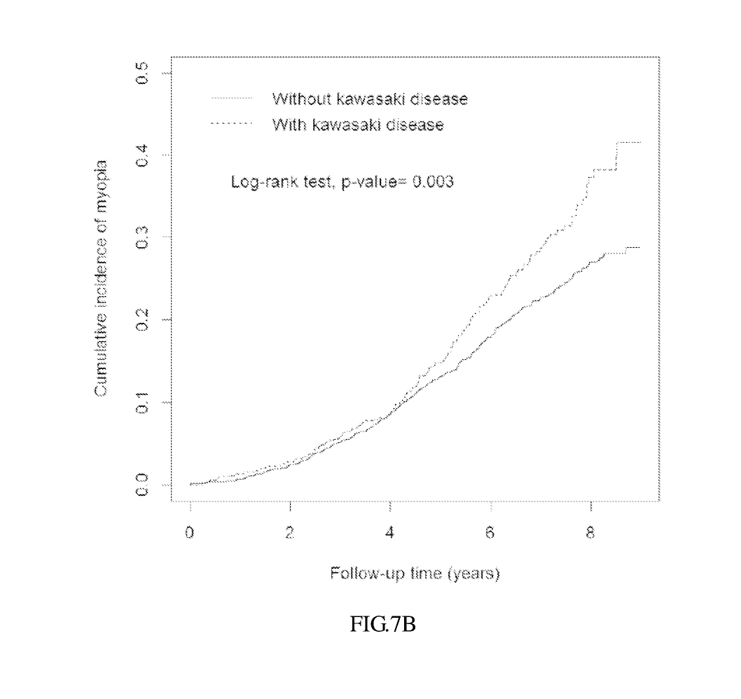

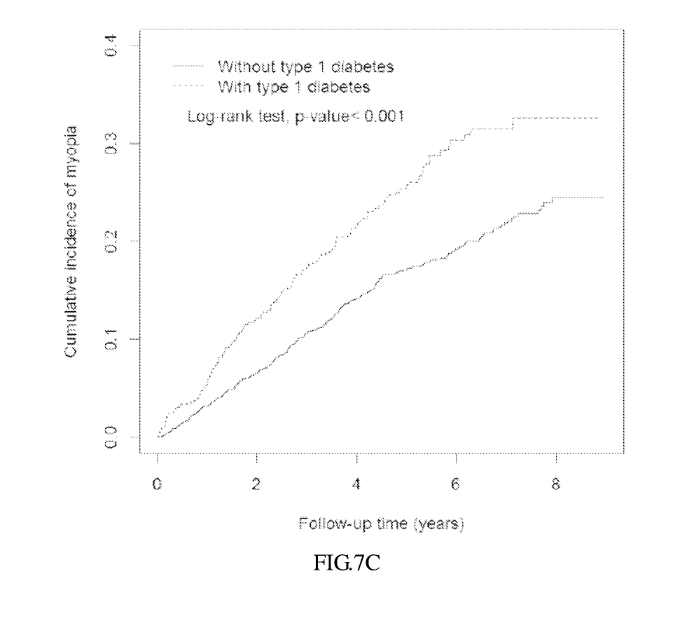

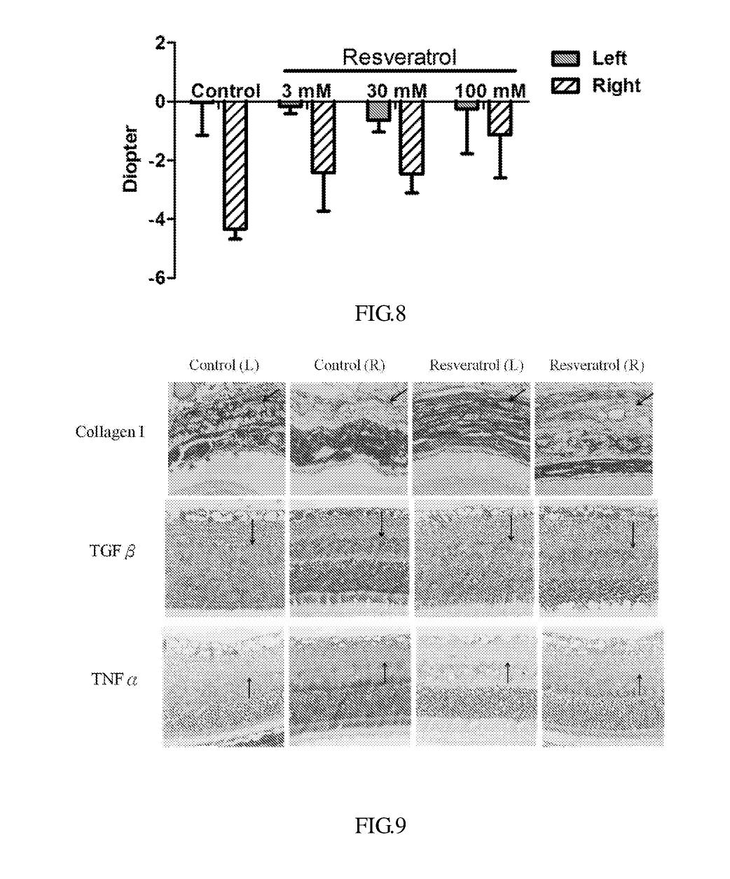

As shown in FIG. 9, the protein expression of collagen I of the right stitched eye is lower than that of the unstitched left eye in the sclera. After 100 mM resveratrol administration, collagen I has been recovered in the sclera. In the control group, TGF-β expression in the retina of the stitched right eye is higher than that of the unstitched left eye, however, TGF-β expression has reduction effect after the administration of 100 mM resveratrol. TNF-α expression in the retina follows the same trend as TGF-β.

### Example 9

### Effect of Resveratrol to the Expression of LPS-Induced Monocyte Chemotactic Protein-1

Human retinal pigment epithelial cells were as model cells under LPS stimulation for 24 hours to induce inflammation and monocyte chemoattractant protein-1 (MCP-1), and then resveratrol was administered to detect the effect of suppressing inflammatory response.

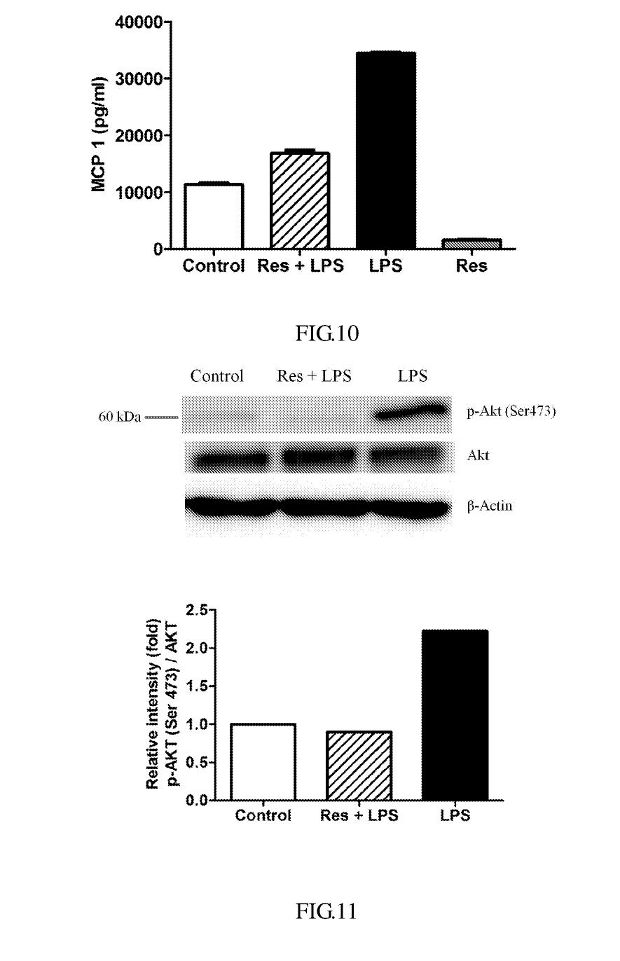

As shown in FIG. 10, 1 μg/ml LPS was used to induce inflammation, such that the expression of MCP-1 was increasing; while the administration of 50 μM resveratrol and 1 μg/ml LPS for 24 hours, the expression of MCP-1 was decreasing, so that resveratrol has significant inhibitory effect on MCP-1.

### Example 10

### Effect of Resveratrol to LPS Stimulation Pathway

Human retinal pigment epithelium was administrated 500 ng/ml LPS for 30 minutes to observe the expression of Akt and ERK.

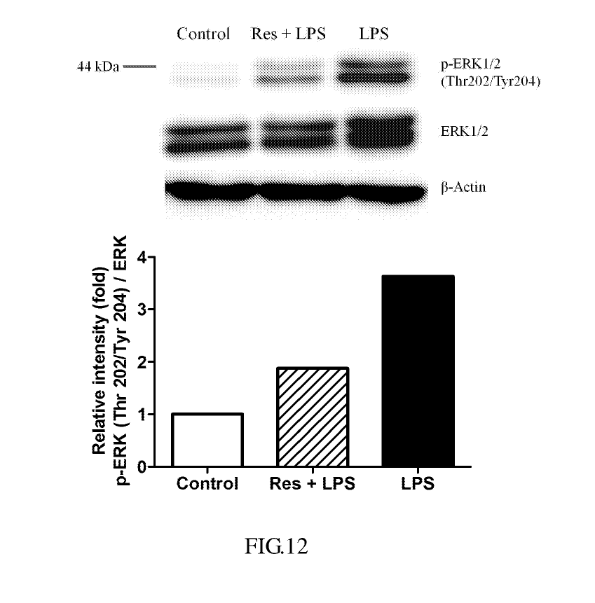

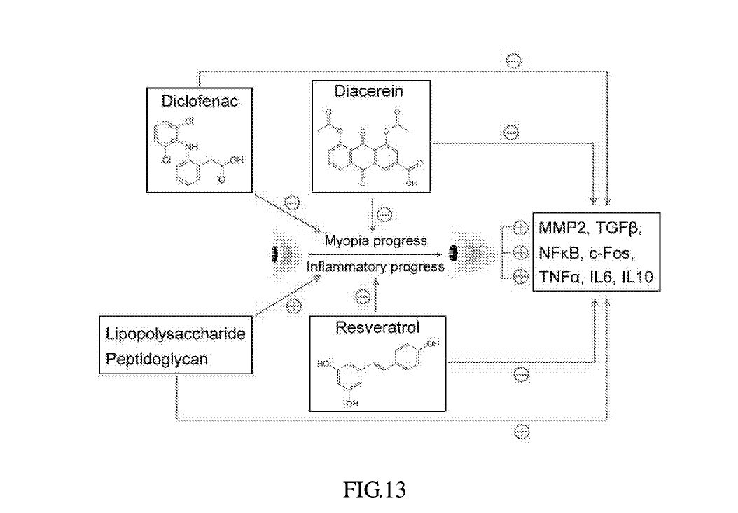

As shown in FIGS. 11 and 12, 500 ng/ml LPS can stimulate the expression of phosphorylated Akt and ERK increasing. Whereas 50 μM resveratrol pretreatment and then 500 ng/ml LPS stimulation, the expression of phosphorylated Akt and ERK would decrease.

### Example 11

### Effect of Diclofenac Administration to Myopia

Four weeks LEWIS rats were used in this example. The right eye induced myopia by FDM were divided into three groups: control group (no administered the drug) and diclofenac solution prepared from Example 14 (6 mg/ml and 3 mg/ml). The refractive error and axial length of right eye were measured by refractometer within three weeks and recorded in Table 14.

TABLE 14
Differences in refractive error after diclofenac treatment

| Variables | Control | 3X diclofenac | 6X diclofenac |
|---|---|---|---|
| Δ Refractive error (D) | −9.20 ± 6.07 | 5.36 ± 4.05* | 1.33 ± 3.30* |
| Δ Axial length (mm) | 1.41 ± 0.68 | 1.04 ± 0.39 | 1.24 ± 0.55 |

Data were the mean ± standard error.
Δ meant the difference between the parameter before and after treatment.
Groups were compared by using t-test where p < 0.05 indicated a statistically significant difference.
*indicated significant difference when compared to Control group.

The two groups administrated with diclofenac solution were all positive two, which means no myopia; the refractive error of the group without any administration (control group) was −9.20 D (severe myopia). The long axial length of eye means severe myopia. The results can also be observed that the axial length of the control group was significantly longer than the two groups administrated with diclofenac solution. In summary, diclofenac administration had a significant inhibitory effect on myopia process. In addition, the inhibitory effect on myopia is better in the concentration of 3 mg/ml than in 6 mg/ml.

The present invention shows the relevance of inflammation and the development of myopia, wherein atropine, although having side effects (such as photophobia and cycloplegia), can inhibit the development of myopia. In addition, the composition of the present invention comprising resveratrol, diacerein or diclofenac (anti-inflammatory agents), and a pharmaceutically acceptable carrier at a specific ratio can be used as an alternative of atropine for inhibiting and/or relieving the progression of myopia.

What is claimed is:

1. A pharmaceutical composition for use in treating and/or relieving myopia comprising: a therapeutically effective amount of an anti-inflammatory agent and a pharmaceutically acceptable carrier, wherein the anti-inflammatory agent comprises resveratrol, diacerein or diclofenac.

2. The pharmaceutical composition of claim 1, wherein the pharmaceutically acceptable carrier comprises potyoxyethylene castor oil ether (Cremophor EL), alkyldimethylbenzylammonium (BKC), lecithin, cholesterol, Dulbecco's phosphate buffered saline (DPBS), cyclodextrin, tween 80, castor oil, artificial tears, and any combination thereof.

3. The pharmaceutical composition of claim 1, wherein the anti-inflammatory agent is resveratrol, the concentration of resveratrol is between 0.1 M and 0.2 M, and the pharmaceutically acceptable excipient comprises cyclodextrin and artificial tears.

4. The pharmaceutical composition of claim 3, wherein the pharmaceutical composition comprises 0.1141 g resveratrol, 1.46 g cyclodextrin and 1 ml artificial tears.

5. The pharmaceutical composition of claim 1, wherein the anti-inflammatory agent is diacerein, the concentration of diacerein is between 0.01 M and 0.02 M, and the pharmaceutically acceptable excipient comprises tween 80, castor oil and artificial tears.

6. The pharmaceutical composition of claim 5, wherein the volume ratio of tween 80 to castor oil is 0.01 to 0.02:0.003 with a total volume of artificial tears.

7. The pharmaceutical composition of claim 6, wherein the pharmaceutical composition comprises 0.004 g diacerein, 1 ml artificial tears, 10 μl tween 80 and 3 μl castor oil.

8. The pharmaceutical composition of claim 1, wherein the anti-inflammatory agent is diclofenac, the concentration of diclofenac is between 0.01 M and 0.02 M, and the pharmaceutically acceptable excipient comprises artificial tears.

9. The pharmaceutical composition of claim 8, wherein the pharmaceutical composition comprises 0.004 g diclofenac and 1 ml artificial tears.

10. A method for preparing said pharmaceutical composition as claimed in claim 1 comprising:
preparing the anti-inflammatory agent;
preparing the pharmaceutically acceptable carrier; and
adding the therapeutically effective amount of the anti-inflammatory agent gradually into the pharmaceutically acceptable carrier under an oscillating condition to obtain the pharmaceutical composition.

11. The method of claim 10, wherein the anti-inflammatory agent is resveratrol, the pharmaceutically acceptable excipient comprises cyclodextrin and artificial tears; the resveratrol is enveloped by cyclodextrin, a molar ratio of resveratrol to cyclodextrin is 1:2 to form a cladding, and the cladding is added to artificial tears at a weight ratio of 0.01 to 0.03:1 progressively and gradely to obtain the pharmaceutical composition.

12. The method of claim 10, wherein the anti-inflammatory agent is diacerein, the pharmaceutically acceptable excipient comprises tween 80, castor oil and artificial tears; the volume ratio of tween 80 to castor oil is 0.01 to 0.02:0.003 with a total volume of artificial tears to form a mixture, and then diacerein is added to the mixture progressively and gradely to obtain the pharmaceutical composition.

13. The method of claim 10, wherein the anti-inflammatory agent is diclofenac, the pharmaceutically acceptable excipient comprises artificial tears; diacerein is added to the mixture progressively and gradely with the weight ratio of diclofenac to artificial tears being 0.03 to 0.06:0.001 to obtain the pharmaceutical composition.

14. A method for treating and/or relieving myopia comprising a step of administering to a subject in a topical in need thereof the therapeutically effective amount of the pharmaceutical composition as claimed in claim 1.

15. The method of claim 14, wherein the subject is animal or human.

16. The method of claim 14, wherein the administration comprises oral administration, topical injection or external use.

17. The method of claim 14, wherein the topical is eyeball.

18. The method of claim 16, wherein the therapeutically effective amount of the pharmaceutical composition for treating and/or relieving myopia in external dosage form is between 0.5% and 1%.

19. The method of claim 16, wherein the therapeutically effective amount of the pharmaceutical composition for treating and/or relieving myopia in oral administration form is between 10 mg/day and 50 mg/day.

20. The method of claim 16, the external dosage form of the pharmaceutical composition is ointments, drops or gels.
